(19) United States
(12) Patent Application Publication
(10) Pub. No.: US 2023/0285227 A1
(43) Pub. Date: Sep. 14, 2023

(54) INGESTIBLE CAPSULES

(71) Applicant: Massachusetts Institute of Technology, Cambridge, MA (US)

(72) Inventors: Shriya Sruthi Srinivasan, Cambridge, MA (US); Carlo Giovanni TRAVERSO, Newton, MA (US); Robert S. LANGER, Newton, MA (US); Amro A. Alshareef, Cambridge, MA (US)

(73) Assignees: Massachusetts Institute of Technology, Cambridge, MA (US); The Brigham and Women's Hospital, Inc., Boston, MA (US)

(21) Appl. No.: 18/182,854

(22) Filed: Mar. 13, 2023

Related U.S. Application Data
(60) Provisional application No. 63/319,620, filed on Mar. 14, 2022.

(51) Int. Cl. A61H 23/02; A61M 31/00

(52) U.S. Cl. CPC A61H 23/0254; A61M 31/002; A61H 2201/1692; A61H 2201/105; A61H 2201/5005; A61H 2205/083; A61M 2205/0238; A61M 2205/10; A61M 2210/106

(57) ABSTRACT

An ingestible capsule includes a housing forming a cavity and having a textured outer surface. The textured outer surface forms a helical depression and a plurality of protruding studs disposed in the helical depression. The capsule further includes a therapeutic agent disposed in or on the housing. The capsule also includes a biodegradable coating on the textured outer surface of the housing, the biodegradable coating configured to dissolve in a fluid having a pH of 1.5 to 9.

### CROSS-REFERENCE TO RELATED APPLICATION

This application claims priority to U.S. Provisional Application No. 63/319,620 titled “VIBRATING INGESTIBLE CAPSULE”, filed Mar. 14, 2022, the entire disclosure of which is incorporated by reference.

### GOVERNMENT SUPPORT

This invention was made with government support under R01 EB000244 awarded by the National Institutes of Health. The government has certain rights in the invention.

### BACKGROUND

Insulin, required daily for millions of diabetic patients globally, is a peptide with oral bioavailability less than 2.5%, necessitating subcutaneous injections, which can lead to injection-related anxiety, pain, and non-adherence. Oral insulin delivery is challenged by poor small intestinal absorption. Amongst other objectives, drugs administered orally do not achieve therapeutic bioavailability unless they 1) overcome the harsh acidic environment of the stomach, 2) dissolve in intestinal fluid, 3) remain stable amongst varying intestinal microbiota, 4) penetrate through the viscous mucus barrier, and 5) evade efflux pumps. Subtherapeutic bioavailability levels pose an unacceptable inefficacy leading many drugs to use alternate, often, more burdensome routes of administration, like intravenous insulin delivery.

Overcoming the hurdles of oral administration poses a large-scale issue for the pharmaceutical industry. Oral administration is the most common, cost-effective, and practical method of drug administration. The ease of oral administration provides higher rates of medication adherence and alleviates any patient anxieties related to injections.

Absorption of orally administered drugs is predominantly challenged by the mucus barrier. Through its viscous, hydrophilic, frequent turnover, and shear-thinning gel properties, mucus serves as a dynamic, steric, and interactive barrier, preventing drugs in the lumen from reaching the epithelial surface.

### SUMMARY

An embodiment of the present technology includes an ingestible capsule. The ingestible capsule includes a housing forming a cavity and having a textured outer surface, a vibrator, vibrating motor, or piezoelectric vibrating component disposed in the cavity, a power supply disposed in the cavity and configured to power the vibrator, a therapeutic agent disposed in or on the housing, and a biodegradable coating disposed on the textured outer surface of the housing. The biodegradable coating is configured to dissolve in a fluid having a pH of 1.5 to 9, thereby exposing the therapeutic agent. The ingestible capsule may also include an electrical resistance component (resistor) with a resistance of about 0 ohms to about 120 ohms.

The ingestible capsule may also include a biodegradable insulating membrane disposed in electrical series between the vibrator and the power supply and in fluid communication with an exterior of the housing. The biodegradable insulating membrane may be configured to dissolve in a fluid having a pH of about 2 to about 9, thereby closing a circuit connecting the power supply and the vibrator. The biodegradable insulating membrane may be configured to dissolve in a fluid having a pH of about 6 to about 7.4.

The textured outer surface of the ingestible capsule may include at least one of a protrusion or depression and may have many different types of textures (e.g., regions with different types, densities, and/or arrangements of protrusions and/or depressions). The at least one protrusion or depression may include a helical depression. The at least one protrusion or depression may include a plurality of protruding studs disposed in the helical depression. Each protruding stud in the plurality of protruding studs may have a diameter of about 200 μm to about 800 μm. The at least one protrusion or depression may include a plurality of slits, which may be uniform or varying in size and/or shape.

The biodegradable coating may include gelatin. The vibrator may include a motor having a shaft, and a weight mechanically coupled to the shaft and radially offset from a longitudinal axis of the shaft. The shaft may be configured to rotate about the longitudinal axis of the shaft at a frequency of about 2 Hz to about 400 Hz (e.g., at about 80 Hz). The power supply may include an energy-harvesting mechanism, chemically charged power supply, wirelessly charged power supply, lithium-ion micro-battery, or silver oxide battery. A silver oxide battery, for example, may have a capacity of about 80 mAh.

Another embodiment of the present technology includes an ingestible capsule. The ingestible capsule includes a housing forming a cavity, a therapeutic agent disposed in or on the housing, and a biodegradable coating on the textured outer surface of the housing. The housing has a textured outer surface. The textured outer surface forms a helical depression and a plurality of protruding studs disposed in the helical depression. The biodegradable coating is configured to dissolve in a fluid having a pH of 1.5 to 9.

The capsule's textured outer surface may include a plurality of slits. The ingestible capsule may include a motor disposed in the cavity and having a shaft, and a weight mechanically coupled to the shaft. The weight may be radially offset from a longitudinal axis of the shaft. The ingestible capsule may include a battery disposed in the cavity and electrically coupled to the motor and, optionally, an inline resistor.

Another embodiment of the present technology includes a method of delivering a therapeutic agent to a subject. The method includes moving a portion of luminal mucus in the small intestine with an ingestible capsule by radially oscillating the ingestible capsule about a longitudinal axis of the ingestible capsule at a frequency of about 50 Hz to about 120 Hz. The method also includes, while moving the portion of luminal mucus, delivering a therapeutic agent from the ingestible capsule to the small intestine.

The method may include dissolving a biodegradable coating disposed on at least part of the ingestible capsule with stomach fluid, water, or an ingested liquid. The method may also include orally ingesting the ingestible capsule.

Another embodiment of the present technology includes an ingestible capsule. The ingestible capsule includes a housing forming a cavity, a vibrator disposed in the cavity, a power supply disposed in the cavity, an optional resistor in series with the power supply, a therapeutic agent disposed in or on the housing, a biodegradable coating disposed on the textured outer surface of the housing, and an insulating membrane disposed in electrical series with the vibrator and the power supply. The housing has a textured outer surface. The textured outer surface forms a helical depression and a plurality of protruding studs disposed in the helical depression. The vibrator is configured to oscillate radially about a longitudinal axis of the ingestible capsule at a frequency of about 50 Hz to about 120 Hz. The radial oscillations cause the ingestible capsule to rotate. The power supply is configured to power the vibrator. The biodegradable coating is configured to dissolve in a fluid having a pH of 1.5 to 3.5. The insulating membrane is in fluid communication with an exterior of the housing. The insulating membrane is configured to dissolve in a biological fluid, thereby closing a circuit connecting the power supply and the vibrator to initiate oscillation.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally and/or structurally similar elements).

FIG. 1A is a schematic illustration of an ingestible capsule.

FIG. 1B is a schematic illustration depicting an actuation mechanism.

FIG. 2 is another schematic illustration of an ingestible capsule.

FIG. 3 is a photograph of an ingestible capsule.

FIG. 4A is a force diagram of an ingestible pill with a rotating motor weight.

FIG. 4B is another view of the force diagram in FIG. 4A.

FIG. 5 shows steps to assemble an ingestible capsule.

FIG. 6A is a schematic illustration of an ingestible capsule having a helical pattern on the outer surface of the capsule.

FIG. 6B shows a photograph of the capsule in FIG. 6A.

FIG. 7A is a schematic illustration of an ingestible capsule having a studded pattern on the outer surface of the capsule.

FIG. 7B shows a photograph of the capsule in FIG. 7A.

FIG. 8A is a schematic illustration of a cross-section of a helical pattern on the outer surface of an ingestible capsule.

FIG. 8B is a schematic illustration of the ingestible capsule having the helical pattern in FIG. 8A.

FIG. 9A is a schematic illustration of a cross-section of another helical pattern on the outer surface of an ingestible capsule.

FIG. 9B is a schematic illustration of the cross-section of an ingestible capsule having the helical pattern in FIG. 9A.

FIG. 9C is a schematic illustration of the ingestible capsule having the helical pattern in FIG. 9A.

FIG. 10A is a schematic illustration of a cross-section of another helical pattern on the outer surface of an ingestible capsule.

FIG. 10B is a schematic illustration of the cross-section of an ingestible capsule having the helical pattern in FIG. 10A.

FIG. 10C is a schematic illustration of the ingestible capsule having the helical pattern in FIG. 10A.

FIG. 11 shows a capsule ingested and then disposed in the small intestine.

FIG. 12 shows barriers to drug absorption in the small intestine.

FIG. 13 shows dissolution of an outer coating on an ingestible capsule having a therapeutic agent.

FIG. 14 is a photograph of an ingestible capsule having a therapeutic agent.

FIG. 15A show a first view of an ingestible capsule with a therapeutic agent.

FIG. 15B shows a cross-sectional view of the ingestible capsule in FIG. 24A.

FIG. 16A shows a capsule with a microtextured outer surface rotating against the inner wall of the small intestine.

FIG. 16B shows helical surface grooves in the microtextured capsule in FIG. 16A gliding and scraping mucus from villi in the small intestine.

FIG. 16C shows studs in the microtextured capsule in FIG. 16A wicking mucus in the small intestine.

FIG. 16D shows release of a therapeutic agent from the microtextured capsule in FIG. 16A.

FIG. 17A shows rotation rates of microtextured capsules having different surface geometries on swine small intestine.

FIG. 17B shows rotation rates of microtextured capsules having different surface geometries in different media.

FIG. 18A shows optical absorbance of luminal fluid in a 4 cm segment of the intestine following 30 minutes of treatment with a microtextured capsule having studs of varying height.

FIG. 18B shows optical absorbance quantification of mucus adhered to microtextured capsules following 30 minutes of rotation in swine small intestine with varying stud height

FIG. 19 shows mixing of a drug in a reaction chamber with a microtextured capsule at varying frequencies.

FIG. 20A shows drug permeabilities for vancomycin delivery with a smooth capsule (control) or microtextured capsule with flat or helical surface geometries in a Franz cell experiment on small intestinal swine tissue.

FIG. 20B shows drug permeabilities for vancomycin delivery normalized to their matched pair in the data shown in FIG. 20A.

FIG. 20C shows drug permeabilities for vancomycin delivery in swine small intestine by smooth capsule (control) or a helical or flat microtextured capsule.

FIG. 20D shows drug permeabilities for vancomycin delivery normalized to their matched pair in the data shown in FIG. 20C.

FIG. 21A shows plasma glucose measurements in swine following luminal insulin (control, upper trace) or via microtextured capsule (experimental, lower trace).

FIG. 21B shows insulin concentration in blood measured after treatment with luminal insulin (control, left) or insulin via microtextured capsule (experimental, right).

### DETAILED DESCRIPTION

The gastrointestinal capsules (also referred to herein as the capsules, ingestible capsules, or pills) disclosed here provides mechanical and neural stimulation within the gastrointestinal tract. In one example, a capsule may be deployed in a subject's gastrointestinal tract orally by the subject ingesting the capsule. In another example, a capsule may be deployed in the gastrointestinal tract by inserting the capsule into the gastrointestinal tract via endoscope or colonoscope. A capsule may also be placed into the stomach via a percutaneous gastrostomy tube (PEG tube). A capsule may mechanically stimulate any desired region within the gastrointestinal tract, including, for example, the stomach, small intestine, and/or large intestine. An exemplary capsule sinks through gastric or other luminal contents and sustains contact with the gastrointestinal lining because of its total weight and density (e.g., a density greater than 2 g cm⁻³). Preferably, the mechanical stimulations provided by the capsule are applied to a portion of the inner walls or lining of a section of the gastrointestinal tract. In one embodiment, the capsule provides mechanical stimulation of the intestinal mucosal barrier to clear mucus for the delivery of a therapeutic agent or drug. An exemplary capsule is naturally evacuated with the stool without obstruction, perforation, or distress. An exemplary capsule may only include low-cost components, so it does not need to be reacquired post evacuation. Similarly, an exemplary capsule need not be recharged.

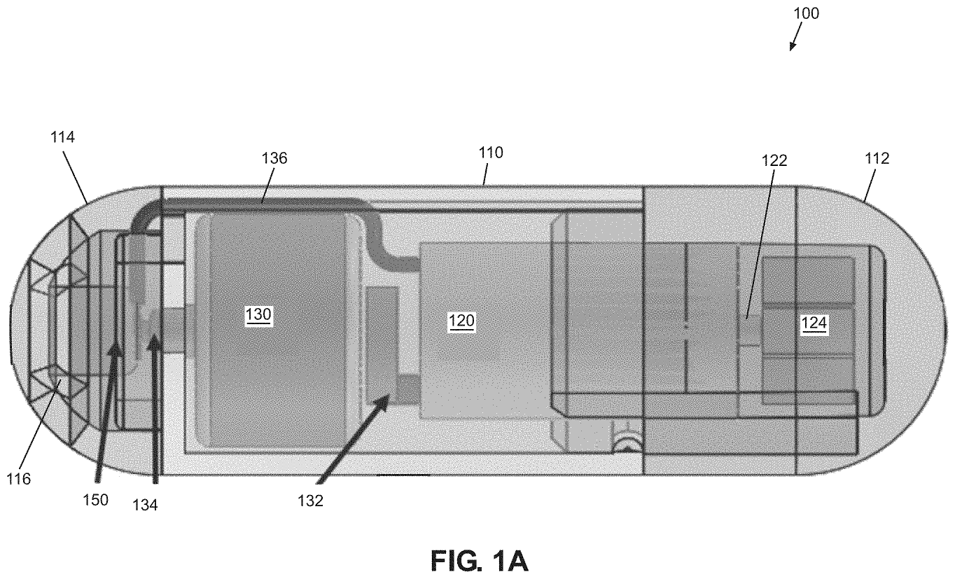

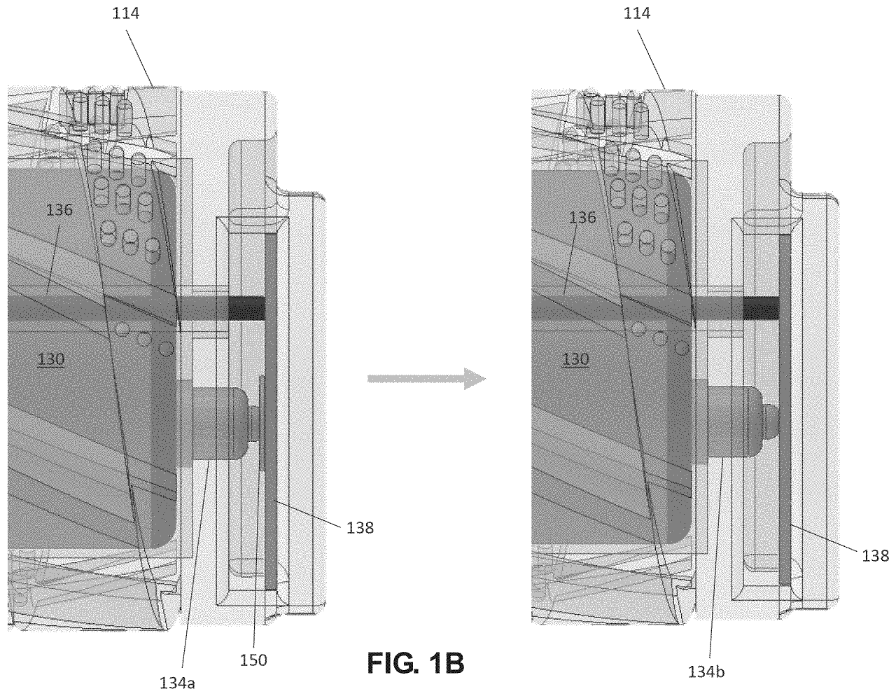

FIG. 1A shows a gastrointestinal capsule 100 that provides mechanical stimulation within the gastrointestinal tract. The gastrointestinal capsule 100 includes a capsule housing including a middle housing 110 and end caps 112 and 114 coupled to opposite ends of the middle housing 110. The capsule housing 110 creates a sealed main cavity in which electronic components are protected from any fluids that the capsule 100 encounters. Inside the main cavity are a motor 120 and a battery 130 or other power supply, such as an energy-harvesting mechanism or wirelessly or chemically charged power supply, configured to provide power to the motor 120. The motor 120 rotates a shaft 122 mechanically coupled to the motor 120. A weight 124 is attached to the shaft in a position so that its center of mass is centered on or laterally offset from the central longitudinal axis of the shaft. The distance between the weight 124 and the motor 120 is limited so as to reduce the total volume of the capsule, but long enough so that the motor body does not interfere with the rotation of the weight. As an example, the weight 124 may have a semi-circular shape with a radius of 2.5 mm. The battery 130 is electrically coupled to the motor 120 in electrical series. A resistor 132 may be electrically coupled in series with the motor 120 and the battery 130 to drop the voltage supplied from the battery 130 to the motor 120. A spring 134 (e.g., a pogo pin, a compression spring, a spring clip, or other spring-loaded connector), which may be conductive or non-conductive, and one or more conductive connectors 136 including wires (e.g., rubber coated copper wires) or cables may also be part of the electrical circuit in the capsule 100.

When the motor 120 is operated, the shaft 122 rotates, causing the weight 124 to also rotate. The motor may be a miniature coreless motor. Coreless motors are preferable because of their high efficiency, high acceleration rates, low inertia, and high power to size ratio. The movement of the weight 124 within the capsule causes capsule movement. Depending on the frequency of rotation, the size of the weight, the type of media that the capsule is in, and the type of surface that the capsule is disposed on, the capsule may move in one or more different ways, including rocking, sweeping, rotating, oscillating, vibrating, and/or teeter-tottering. For example, when the capsule 100 is in contact with a plical surface, it will rotate. If the capsule 100 is unconstrained on its sides, it will rock back and forth. The pattern of capsule movement also changes when the capsule 100 is in contact with bumps and/or grooves in the tissue. These capsule movements provide mechanical stimulation to a portion of tissue within the gastrointestinal tract.

The placement of the weight within the capsule also determines the type of capsule movement. For example, the center of mass of the weight may be centered on or laterally offset and/or longitudinally offset from the center of the capsule. In a version, the center of mass of the weight is laterally offset from the central longitudinal axis of the capsule by about 1 mm to about 2 mm. In the same or a different version, the center of mass of the weight is centered on the lateral axis or longitudinally offset from the center of the capsule up to the edge of the capsule's end cap (e.g., 1 mm, 2 mm, 3 mm, 4 mm, 5 mm, 6 mm, 7 mm, 8 mm, 9 mm, 10 mm, 11 mm, 12 mm, or 13 mm from the center of the capsule). In a version, the center of mass of the weight is longitudinally offset from the center of the capsule by about 11.2 mm. When the center of mass of the weight is longitudinally offset, rotation of the weight about the motor's shaft creates a teeter-totter motion in the capsule. The distance of longitudinal offset determines the amplitude of the teeter-totter motion. In a version, one end cap of the capsule is mechanically coupled to the motor shaft and the other end cap is mechanically coupled to the motor body so that both end caps rotate relative to each other at a rate proportional to their respective masses, so that the two sides of the capsule rotate in opposite directions to facilitate mixing.

In some embodiments of the capsule, the outer casing of the motor itself can serve as the outer shell, or a portion of the outer shell, of the capsule. This would allow for a smaller size capsule by eliminating an additional layer over the motor. Having the outer casing of the motor serves as at least a portion of the capsule's outer shell may also facilitate relative rotation of the capsule's end caps.

The weight rotation frequency may be about 2 Hz to about 400 Hz (e.g., 2 Hz, 5 Hz, 10 Hz, 20 Hz, 30 Hz, 40 Hz, 50 Hz, 60 Hz, 70 Hz, 80 Hz, 100 Hz, 120 Hz, 150 Hz, 180 Hz, 200 Hz, 250 Hz, 300 Hz, 350 Hz, or 400 Hz). Preferably, the frequency is about 60 Hz to about 120 Hz (e.g., about 80 Hz). Operation of the motor at these frequencies over the operational time period does not generate enough heat to cause any tissue damage. Operation of the motor at these frequencies also does not cause abrasion, irritation, or inflammation of the tissue. As an example, operation of the motor may cause capsule displacement amplitudes of about 0 mm to about 5 mm when powered with a 1.55 V silver oxide battery.

FIG. 1B shows an actuation assembly in one embodiment of the capsule 100. In this embodiment, the circuit is completed and the motor 120 begins receiving power when a membrane 150 (or a thin layer, barrier, coating, film, or sheet) is dissolved or degraded. The left image in FIG. 1B shows the actuation assembly in the pre-actuation state where the motor 120 is not receiving power. The right image in FIG. 1B shows the actuation assembly in the actuated state where the electrical circuit is completed and the motor 120 is receiving power. In the left image in FIG. 1B, the spring in the pogo pin 134a is compressed and a surface of the pogo pin's plunger is in direct contact with a surface of the membrane 150. The membrane 150 is disposed between the pogo pin's plunger and a mating receptacle 138 (e.g., a target or a land, having a flat or concave conductive surface) for the pogo pin 134b to engage, to complete the connection path when the membrane 150 is no longer present. When the membrane 150 degrades or dissolves, the pogo pin plunger extends under the force of the pogo pin's spring to contact the mating receptacle 138. A conductor 136 electrically connects the mating receptacle 138 to the battery 130. If desired, a capacitor (not shown) can form an RC delay element with the resistor 132 that introduces a time delay to offset the activation of the motor 120 in response to the conductor 136 contacting the mating receptacle 138.

The membrane 150 degrades or dissolves over a desired period of time when in contact with fluid in a particular pH range in order to complete the circuit to the motor 120. For example, the desired period of time may be about 1 minute to about 2 hours (e.g., 1 minute, 2 minutes, 3 minutes, 4 minutes, 5 minutes, 6 minutes, 7 minutes, 8 minutes, 9 minutes, 10 minutes, 20 minutes, 30 minutes, 1 hour, or 2 hours). In one version, the membrane 150 dissolves in about 5 minutes (e.g., 4.3±1.2 minutes), soon after it contacts the fluid in the set pH range. The set pH range may be about 2 to about 9, or any sub-range therein. In one version, the pH range is about 1.5 to about 3.5, so that the membrane 150 degrades or dissolves, for example, when it contacts gastric fluid. In another version, the pH range is about 6 to about 7.4, so that the membrane 150 degrades or dissolves, for example, when it contacts fluid in the small intestine. In another version, a medication or other co-administered agent induces a pH of about 7 to about 9 in a desired region of the gastrointestinal tract, and the membrane 150 degrades or dissolves in fluids in this pH range to activate the capsule in this desired region. The membrane 150 may be insoluble or have a sufficiently slow dissolution/degradation rate in fluids outside the set pH range to prevent capsule activation when the capsule is in a fluid outside the set pH range. The thickness of the membrane 150 may be selected so that it dissolves or degrades in a desired amount of time. The membrane 150 may have a thickness of about 0.5 mm to about 5 mm. Preferably, the membrane 150 has a thickness of about 0.5 mm to about 2.5 mm. More preferably, the membrane 150 has a thickness of about 0.5 mm to about 1 mm.

The membrane 150 may be a polymer with pH sensitive chemical bonds that are cleaved in a particular pH range. For example, the cleavable bonds may include imine bonds, hydrozone bonds, oxime bonds, amide bonds, acetal bonds, orthoester bonds, acrylate bonds, and/or methacrylate bonds. The membrane 150 may include glucose, gelatin, chitosan, Eudragit, poly lactic-co-glycolic acid (PLGA), polylactic acid (PLA), polycarbonate (PC), polycarboxylic acid (PCA), polyglycolide (PGA), and/or polymethacrylate. Preferably, the membrane 150 includes Eudragit. The membrane 150 may be a biocompatible material so that when it degrades or dissolves inside the body, it is not harmful to living tissue.

In one example, the membrane 150 may be a discrete shape (e.g., circle or rectangle) that is as small as the diameter of the pogo pin's plunger or as big as the capsule 100 itself. In another example, the membrane 150 may be part of a layer or coating disposed on the exterior surface of the capsule's housing.

In one example, the membrane 150 may be disposed in a cavity formed in the end cap 114. This end cap cavity is in fluid communication with fluid outside of the capsule 100. For example, the end cap 114 may have one or more openings or conduits 160 in the end cap 114 so that fluid can move between the interior of the end cap cavity and the exterior of the capsule 100. The main cavity is sealed off from the end cap cavity with medical-grade adhesive or sealant so that fluid does not enter the main cavity. At least part of the pogo pin 134 plunger and the mating receptacle 138 are disposed in the end cap cavity. In another version, the membrane 150 and portions of the pogo pin 134 and the mating receptacle 138 are disposed on an exterior surface of the capsule 100 (e.g., the exterior of the central housing), where they freely interact with fluid in the environment of the capsule 100. For example, the seal may be formed by filling the connection points and wire/pogo pin through-holes in the main cavity with a seal that is impermeable to GI fluids (e.g., medical grade epoxy). The pogo pin 134 plunger and the mating receptacle 138 may be made of one or more biocompatible conductive materials, including gold, platinum, or palladium. The pogo pin 134 and the mating receptacle may also be chemically resistant to gastrointestinal fluids.

Alternatively, the capsule 100 may include one or more sensors (not shown), such as an accelerometer, temperature sensor, pH sensor, or piezoelectric sensors, instead of a dissolvable membrane. When the sensor senses when the capsule 100 has entered the desired region of the gastrointestinal tract, it triggers the motor 120. The capsule 100 could also include a wireless receiver or antenna that receives a wireless signal, such as a Bluetooth low energy (BLE) signal, from a device outside the body and triggers the motor 120 in response to the signal.

The battery 130 may be a primary battery that provides power to the motor for up to about 2 hours. For example, the battery 130 may power the motor for 10 minutes, 20 minutes, 30 minutes, 40 minutes, 50 minutes, 1 hour, 1.5 hours, or 2 hours. Preferably, the battery 130 powers the motor for about 30 minutes to about 40 minutes. The battery 130 may be a silver oxide battery, a lithium battery, a copper-zinc battery, or a zinc-carbon battery. The battery may supply a voltage of 1.55 volt (V) to about 3 V. For example, the battery may supply a voltage of 1.55 V, 1.60 V, 1.65 V, 1.7 V, 1.8 V, 1.9 V, 2.0 V, 2.2 V, 2.5 V, 2.8 V, or 3.0 V. Preferably, the battery 130 is a silver oxide battery, which is very biocompatible and used in several FDA-approved devices, with a voltage of 1.55 V. The battery may have a capacity of 30 milliamp-hours (mAh) to about 300 mAh (e.g., 30 mAh, 50 mAh, 80 mAh, 100 mAh, 150 mAh, 200 mAh, 250 mAh, or 300 mAh), and the capacity may be chosen depending on the desired operation time of the motor 120 and the size of the pill (capsule). In one example, the silver oxide battery has a capacity of 80 mAh and operates for about 30 minutes to about 40 minutes. The electrical circuit may include a resistor 132 to drop the voltage supplied to the motor 120 in order to control the motor's frequency. The resistor 132 may have a resistance between about 0 ohms and about 10,000 ohms (e.g., 120 ohms).

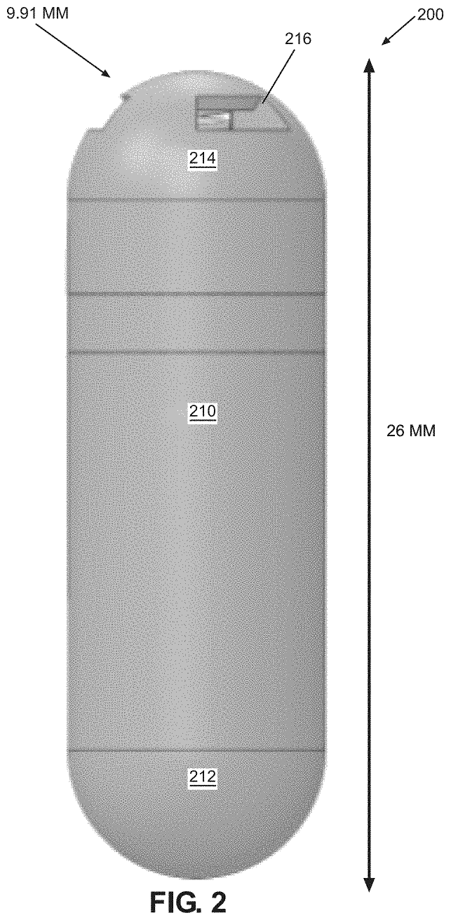

FIG. 2 shows the outer housing of a gastrointestinal capsule 200. The capsule 200 includes middle housing 210 and end caps 212 and 214 coupled to opposite ends of the middle housing 210. The capsule size may be zero (0), double zero (00), or triple zero (000). In one example, the capsule 200 is a triple-zero (000) capsule, with a length of about 26 mm and a diameter of about 9.91 mm. The housing components are rigid, biocompatible, and chemically stable within the environment of the gastrointestinal tract. In some versions, the housing may also be transparent. As an example, the housing material may be VeroClear, a photopolymer that simulates polymethylmethacrylate (PMMA), PMMA, gelatin, hydroxypropyl cellulose, ellastolan, and/or pullulan. The thickness of the housing walls is chosen to provide enough space for the electronic components while still being manufacturable and rigid enough to transmit vibrational force. For example, the thickness may be about 0.4 mm to about 1 mm (e.g., 0.4 mm, 0.5 mm, 0.6 mm, 0.7 mm, 0.8 mm, or 1.0 mm). Preferably, the thickness of the housing wall is about 0.6 mm. The capsule 200 includes a conduit 216 though which gastric fluid, small intestinal fluid, water, or other injected substances may flow to dissolve the membrane in the end cap cavity to activate the capsule's motor. The three housing sections 210, 214, and 216 may be press-fit together to create a tightly sealed main cavity. The three housing sections 210, 214, and 216 may also be sealed together and/or coated with biocompatible adhesive to maintain a fluid-proof seal. In other embodiments, the housing may be a single piece or two pieces instead of three. The housing components may be 3D printed or injection molded (e.g., via two-shot molding or overmolding).

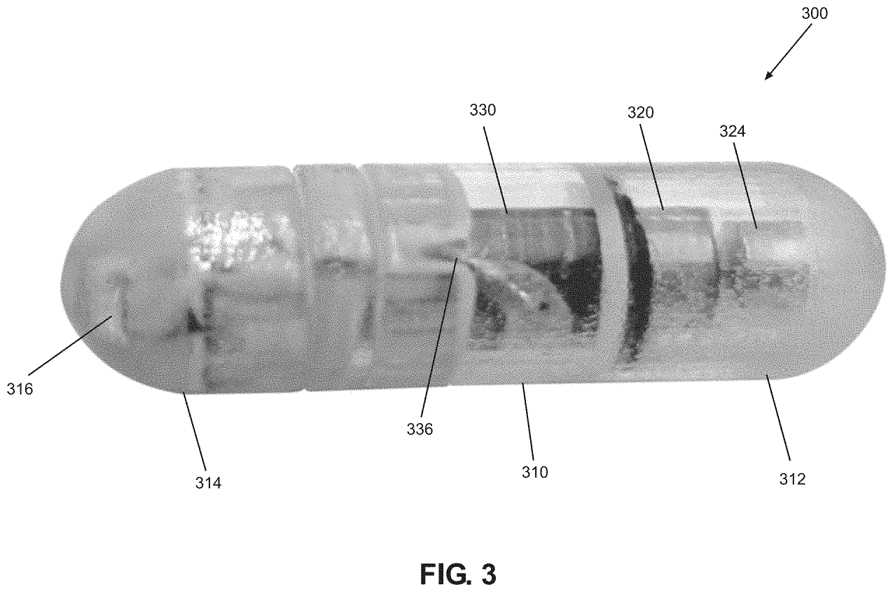

FIG. 3 is a picture of a gastrointestinal capsule 300. The capsule 300 includes a capsule housing including a middle housing 310 and end caps 312 and 314 coupled to opposite ends of the middle housing 310. The capsule housing creates a sealed main cavity in which electronic components are protected from any fluids that the capsule 300 encounters. Inside the main cavity are a motor 320 and a battery 330 configured to provide power to the motor 320. The motor 320 rotates a shaft mechanically coupled to the motor 320. A weight 324 is attached to the shaft in a position so that its center of mass is laterally offset from the central longitudinal axis of the shaft. A conduit 316 in the end cap 314 provides fluid coupling between a separate cavity in the end cap 314 and the external environment of the capsule 300. The capsule 300 includes an actuation assembly that includes a membrane disposed between a conductive spring and a conductive connector 336. The membrane dissolves or degrades when in contact with a fluid of a set pH, thereby closing the electrical circuit and actuating motor operation. The membrane can also be tuned to dissolve to a temperature cue.

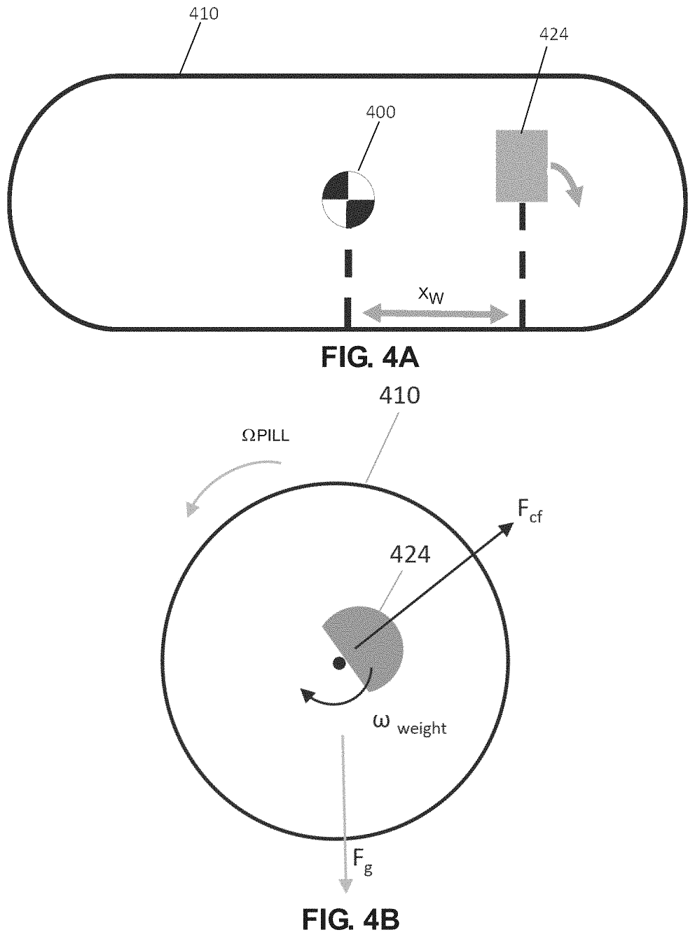

FIGS. 4A and 4B show two different views of a force diagram of an ingestible capsule with a rotating weight 424 offset from the center point of the capsule 400 inside the capsule's housing 410. Rotation of the weight 424 generates a centrifugal force, F_{cf}, causing the capsule housing 410 to move against surface friction. F_{cf} pulls the capsule housing 410 radially outward and changes the capsule housing's direction with the offset weight 424:

$${F}_{cf} = {m}_{weight} ⋆ {ω}_{weight}^{2} ⋆ {r}_{weight} \qquad ( 1 )$$

$$f = \frac{{ω}_{weight}}{2 ⋆ pi} \qquad ( 2 )$$

where ω_{weight} is the angular velocity of the weight 424 and r_{weight} is the radial offset of the weight 424 from the central point 400 of the capsule. The resulting vibrational frequency of the capsule is f. The oscillatory movement of the capsule is caused by the offset of this force to one side of the capsule by x_{w} from the center of mass 400, which causes the capsule to rock as the weight 424 moves with and against the force of gravity.

ΣL=L_{weight}+L_{capsule}=0 (3)

I_{weight}*ω_{weight}=−I_{capsule}*Ω_{capsule} given ω_{weight}>>Ω_{capsule} (4)

The rotational movement of the capsule is governed by conservation of momentum within the system. In a frictionless environment, as the motor within the capsule spins at ω_{weight}, the capsule counters that spin with an angular velocity Ω_{capsule}, proportional to the rotational rate of the motor and scaled by a ratio of the moment of inertia of the weight, I_{weight}, to that of the capsule, I_{capsule}.

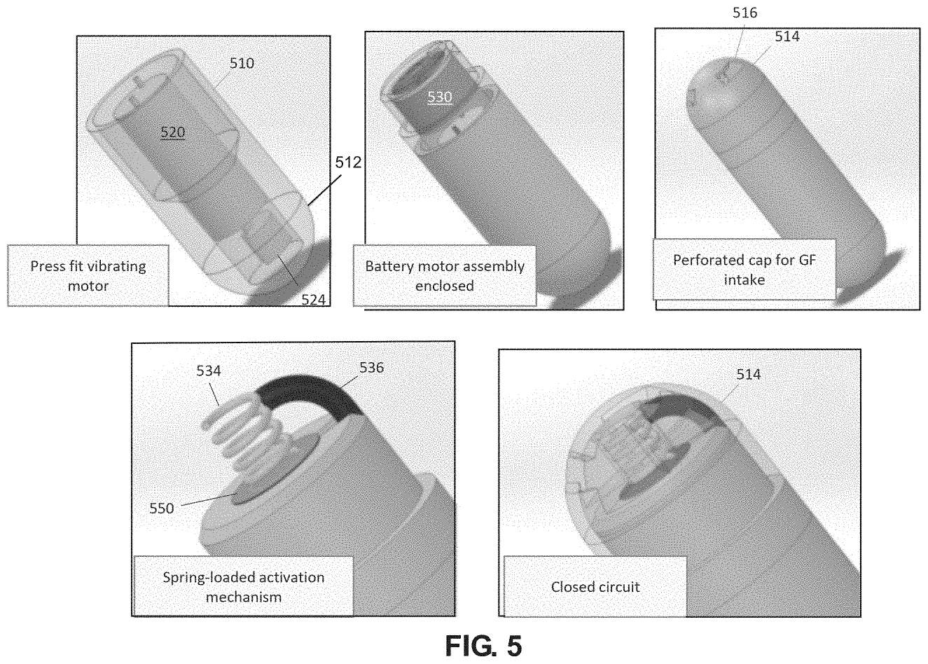

FIG. 5 shows a method of assembling a gastrointestinal capsule. The capsule is split into three sections: the motor cap (or end cap) 512, the central body (or middle housing) 510, and the pill cap (or end cap) 514. The vibrating motor 520, mechanically coupled to an offset weight 524, is first pressed into the motor cap 512. The battery 530 is then placed in the central body 510. A copper pad is soldered on the positive lead of the motor 520 and positioned near the positive lead of the battery 530. The spring loaded pogo pin 534 is then pressed into the central body 510, so that the battery's negative terminal is in contact with the pogo pin 534. The central body 510 assembly is then pressed onto the motor cap 512 assembly such that the positive terminal of the battery 530 contacts the positive copper pad on the motor 520. The negative lead of the motor 520 is stretched through the central body 510 to the pill cap 514. The negative lead is soldered onto a conductive pad (or mating pad) in the top section that when closed onto the pill, closes the circuit by contacting the pogo pin 534. A membrane 550 is also placed between the negative lead pad and the pogo pin 534. The membrane 550 dissolves once the pill reaches the desired gastrointestinal fluid, so that the capsule is activated only when it reaches the desired section of the gastrointestinal tract.

The outer surface of the capsule housing may be smooth or may be microtextured. The surface geometry of the outer surface may be selected for a particular application. For example, microtexturing the surface may increase or reduce drag, and/or increase or reduce capsule rotation. Microtexturing may promote smooth rotations of the capsule about the longitudinal axis. Alternatively, microtexturing may promote capsule rocking movements or vibrations that help the capsule push down into the mucosa.

Microtexturing may include helical patterns (also called spiral patterns), stud patterns (also called nub, bump, or nodule patterns), or slit patterns. The microtexturing may protrude out from the surface of the housing or may intrude into the surface of the housing (e.g., as a groove). The microtexturing may be disposed over the entire outer surface of the housing or on only a portion (e.g., only on the central part of the housing or only on the end caps). More than one type of microtexturing may be included (e.g., both stud and helical patterns). The microtexturing may be formed into the capsule housing or may be a separate layer that is disposed onto the capsule housing. The microtextured housing or microtextured layer may be formed by 3D printing, injection molding, laser cutting, laser grooving, press molding, extrusion, thermoforming, texturing using mills, texturing using abrasive materials, texturing using molding, texturing using polymer casting, and/or blow molding. One-shot or multi-shot molding may be used to form microtextured components.

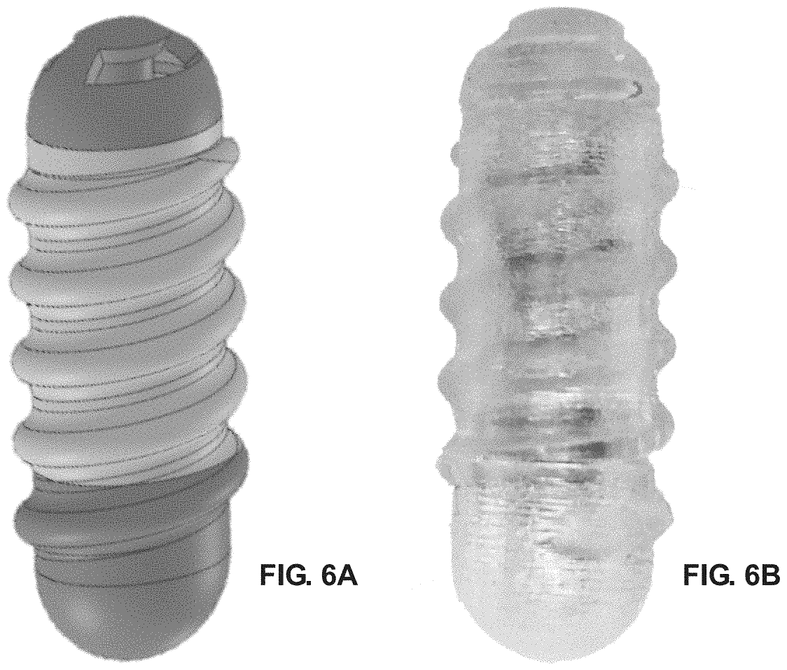

FIG. 6A is a schematic illustration of an ingestible capsule having a protruding helical microtextured pattern on its outer surface. The helical microtextured pattern provides a screw-like motion to facilitate a directional turning and churning motion. FIG. 6B shows a photograph of the capsule in FIG. 6A. The length, width, and angle of the helix may be varied. For example, the width of the helix may be about 0.2 mm to about 8 mm, preferably about 0.5 mm to about 2 mm, more preferably about 1 mm. The pitch of the helix may be about 2 mm to about 9 mm, and preferably about 4.5 mm. The helical pattern may be right-handed or left-handed.

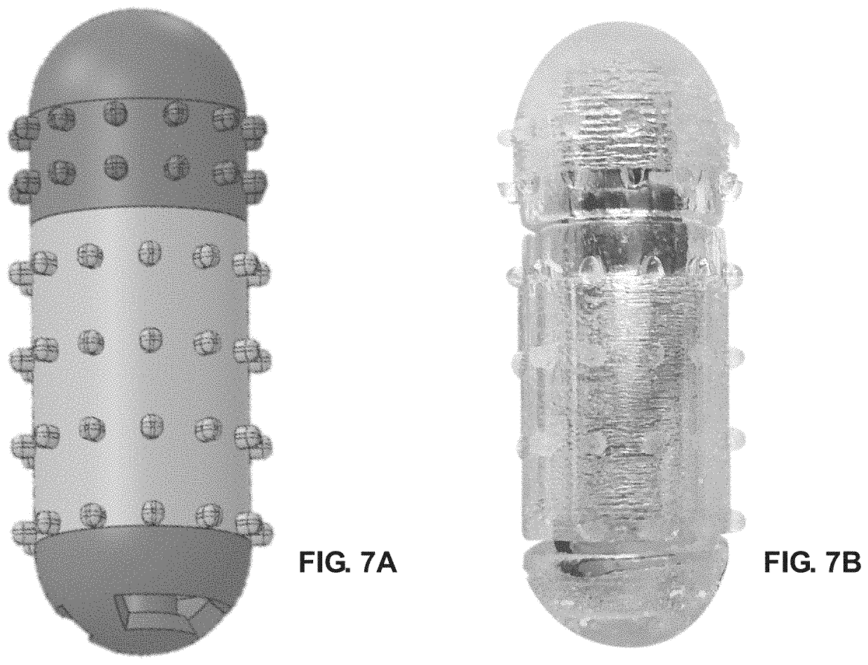

FIG. 7A is a schematic illustration of an ingestible capsule having a studded pattern on its outer surface. The studded pattern (also called protruding nubs) digs into and churns the mucosal layer. FIG. 7B shows a photograph of the capsule in FIG. 7A. The length of the studs extending out from the surface of the capsule may be about 200 μm to about 1200 μm. For example, in a version of the capsule used to wick mucus, studs with a length of about 700 μm to about 900 μm are preferable. The distribution of studs and the number of studs may also be varied. The studs may be distributed radially at 30- to 60-degree increments (e.g., 30 degrees, 45 degrees, or 60 degrees). The studs may be axially distributed at 1 mm to 6 mm increments. Stud patterns may be combined with any other microtexturing pattern. For example, the capsule may have an intruding helical pattern with studs patterned on the intruding helix surface.

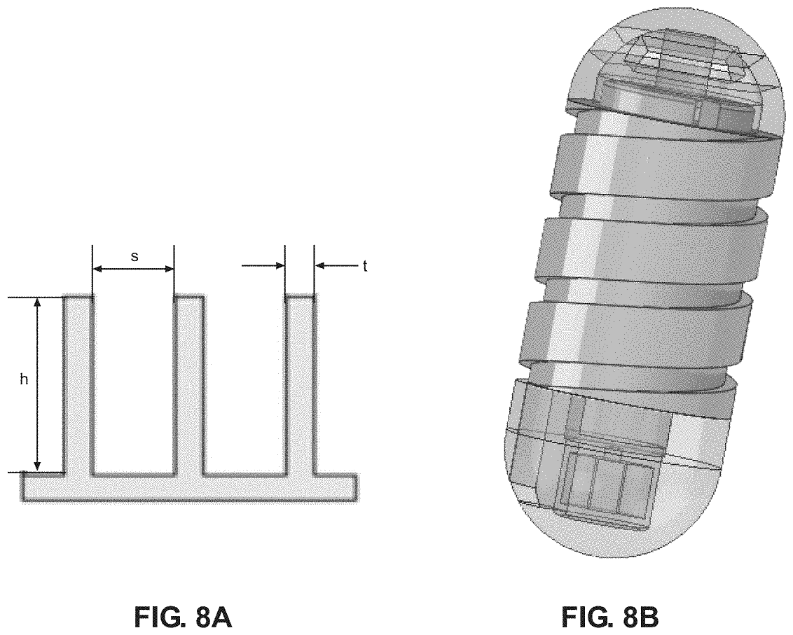

FIG. 8A is a schematic illustration of a cross-section of an intruding helical pattern on the outer surface of an ingestible capsule. FIG. 8B is a schematic illustration of the ingestible capsule having the intruding helical pattern in FIG. 8A. The intruding helical pattern (also called a spiral pattern) serves to reduce contact surface area, thereby reducing friction on the capsule that would counteract the capsule's rotational movement. The length, width, and angle of the helix may be varied. The pitch of the helix may be about 2 mm to about 9 mm, and preferably about 4.5 mm. The width of the helix may be about 0.2 mm to about 4 mm. The depth of the helix may be about 0.1 mm to about 1 mm. The helical pattern may be right-handed or left-handed.

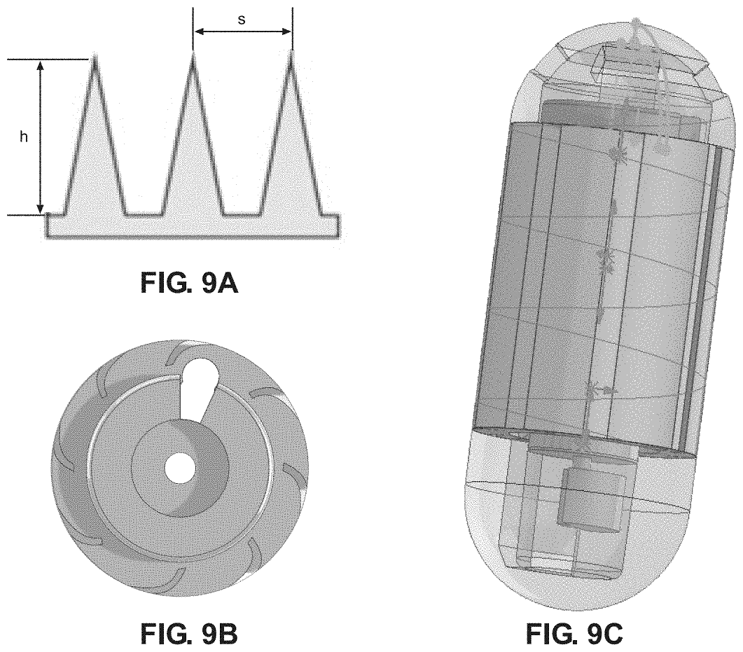

FIG. 9A is a schematic illustration of a cross-section of another intruding helical pattern on the outer surface of an ingestible capsule. FIG. 9B is a schematic illustration of the cross-section of an ingestible capsule having the helical pattern in FIG. 9A. FIG. 9C is a schematic illustration of the ingestible capsule having the helical pattern in FIG. 9A. The fin turbine pattern shown in FIG. 9C provides a churning motion. Like the helical pattern in FIG. 8A, the length, width, and angle of the helix may be varied. Unlike the helical pattern in FIG. 8A, the pattern in FIG. 9A has sawtooth points instead of flat edges that create points of friction that help the capsule rotate and stroke mucosa.

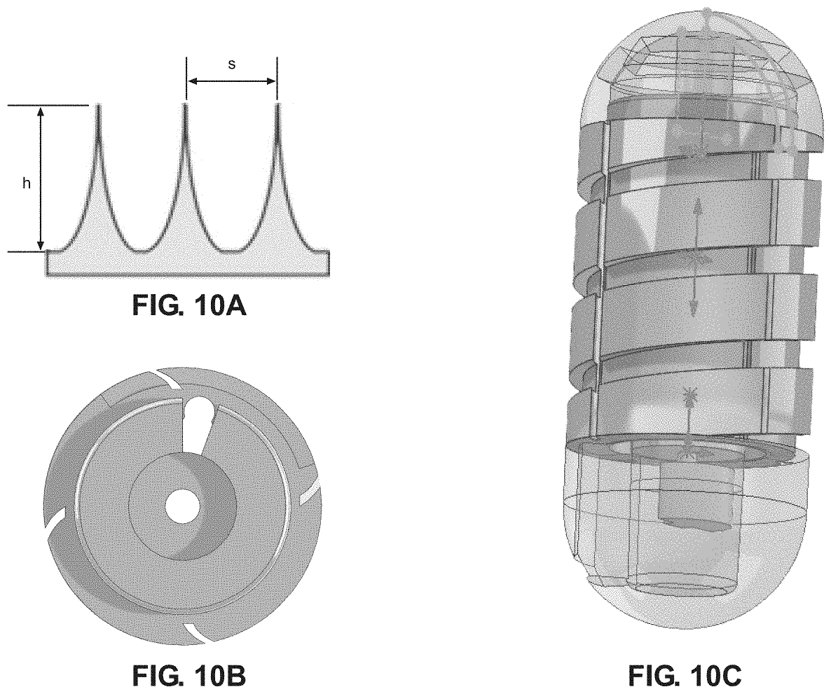

FIG. 10A is a schematic illustration of a cross-section of another helical pattern on the outer surface of an ingestible capsule. FIG. 10B is a schematic illustration of the cross-section of an ingestible capsule having the helical pattern in FIG. 10A. FIG. 10C is a schematic illustration of the ingestible capsule having the helical pattern in FIG. 10A. The helical pattern shown in FIG. 10C provides a combination of benefits, including reducing contact surface area to reduce friction that would counteract the capsule's rotational movement, and providing a churning motion. Like the helical pattern in FIG. 8A, the length, width, and angle of the helix may be varied. Unlike the helical pattern in FIG. 8A, the pattern in FIG. 10A has scalloped points instead of flat edges that create points of friction that help the capsule rotate and stroke mucosa.

The capsule may include a coating disposed on the surface of the housing. In versions of the capsule that include microtexturing, a coating may be disposed over the microtextured surface to cover these features. In this way, the coating may promote safe and comfortable capsule swallowing and passage through the gastrointestinal tract. The coating may degrade or dissolve in a fluid of a set pH in the same way as the membrane in the actuation assembly. The set pH may include any of the ranges described above with respect to the membrane. The coating may be formed from any of the materials described with respect to the membrane. In a version, the coating is the same material or materials as the membrane so that the coating and membrane both dissolve and/or degrade in the desired region of the gastrointestinal tract concurrently. In another version, the coating is a different material than the membrane so that the capsule actuation does not occur until the capsule navigates two regions of the gastrointestinal tract, one region dissolving or degrading the coating and the second dissolving or degrading the membrane. This configuration lends greater control over the actuation sequence within the body. As another benefit, the coating may add a layer of protection to prevent fluid leakage into the main cavity of the capsule where the electronic components are disposed.

The capsule may include a therapeutic agent. Because of the capsule's mechanical stimulation mechanisms, the capsule may be used to deliver therapeutic agents through oral means that are conventionally delivered through non-oral means given the low bioavailability of the oral method. The mechanical stimulation enhances drug delivery through various mechanisms described in more detail below. The therapeutic agent may include small molecular weight drugs, biotherapeutic macromolecules, proteins, and/or nucleic acid-based therapies. For example, the therapeutic agent may include biologically derived therapeutics and/or macromolecular therapeutics having a molecular weight less than about 10 kilodaltons (kDa). As another example, the therapeutic agent may include drugs having a molecular weight less than 10 kDa to about 175 kDa (e.g., less than 10 kDa, 10-25 kDa, 25-50 kDa, 50-75 kDa, 75-100 kDa, or 100-175 kDa). As another example, the therapeutic agent may be a peptide or small protein having a molecular weight less than 10 kDA (e.g., insulin and vancomycin). As another example, the therapeutic agent may be used with class III and/or class IV drugs with low permeability in the intestines per the biopharmaceutical classification system (BCS). A low drug oral bioavailability may be less than 1%, less than 2.5%, less than 5%, and/or less than 10%. Other ranges or thresholds are also possible.

The capsule may be used to deliver a therapeutic agent for any of a wide range of diseases, including infectious diseases, deficiency diseases, hereditary diseases, and physiological diseases. In addition to or alternatively to delivering drugs, the capsule may also deliver micronutrients, vitamins, chemical agents, and/or herbal chemicals.

The therapeutic agent may be loaded into or onto the capsule in any of several ways. The therapeutic agent may be disposed in or on an endcap of the capsule, so that it may be easily accessed and manipulated. The therapeutic agent may be a gel or powder that is pressed into the cavity. Alternatively, the therapeutic agent may be formed into a hard tablet (e.g., in the shape of a hemisphere) that mechanically couples (e.g., via press-fitting) to the middle housing of the capsule, thereby forming the endcap. In this version, the tablet may include one or more excipients used to form the tablet and/or control dissolution and/or erosion of the tablet in the gastrointestinal tract. Alternatively, the endcap may form a separate cavity that is sealed off from the main cavity, and the therapeutic agent may be loaded into the endcap cavity. In this version, the therapeutic agent may be a solid, liquid, gel, or other viscous formulation. The endcap housing or compartment may include one or more conduits to release the therapeutic agent into the gastrointestinal tract, and the conduits may be blocked with a barrier layer or membrane that dissolves or degrades when exposed to a fluid in set pH range. The material of this barrier layer may be any of those described above with respect to the membrane layer in the actuation assembly, and the set pH may be any of the ranges described above with respect to the membrane layer. Alternatively, the therapeutic agent may be coated as a layer on the outer surface of the capsule, and the coating may dissolve or degrade when exposed to a fluid in a set pH range. For example, the therapeutic agent may be mixed into the coating described above.

The amount of therapeutic agent loaded in or on the capsule may have a volume of about 0 mm³ to about 342.6 mm³ (e.g., 50, 100, 150, 200, 250, or 300 mm³). For example, if the therapeutic agent is formed into an endcap tablet, the drug payload volume may be as large as 342.6 mm³. Other ranges of therapeutic amounts are possible, e.g., 400 mm³, 500 mm³, or 600 mm³, with appropriately sized capsules.

Mechanical movement of the capsule facilitates spreading of the therapeutic agent and integration into the mucosa layers in the gastrointestinal tract. Through its mechanical movements, the capsule may press and rub the therapeutic agent into the mucosal layers while also clearing mucus away from the mucosa. If the therapeutic agent is a solid formation, it may erode away layer by layer as it rotates on the mucosa. If the therapeutic agent is a liquid, the liquid is released in a desired location within the gastrointestinal tract and dispersed into the mucosa.

Given the capsule's unique ability to interface with the mucosa, a sensor or multiple sensors may be placed in the endcap instead of or in addition to a therapeutic agent. The sensors may directly sample mucosal conditions. The capsule's microtexture may be selected to increase mucus wicking to access the mucosal layers for sampling. Alternatively, the capsule may not include wicking microtextural patterns and instead sample the mucus itself. The sensor or sensors may measure pH, chemical composition, electrical activity, inertial movement, microbiota. The sensors may also perform optical stimulation or optical sensing.

Example Therapeutic, Diagnostic, and/or Enhancement Agents for Capsule Delivery

According to some embodiments, the capsules and methods described herein are compatible with one or more therapeutic, diagnostic, and/or enhancement agents, such as drugs, nutrients, microorganisms, in vivo sensors, and tracers. In some embodiments, the active substance, is a therapeutic, nutraceutical, prophylactic or diagnostic agent. While much of the specification describes the use of therapeutic agents, other agents listed herein are also possible.

Agents can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals, Certain such agents may include molecules such as proteins, peptides, hormones, nucleic acids, gene constructs, etc., for use in therapeutic, diagnostic, and/or enhancement areas, including, but not limited to medical or veterinary treatment, prevention, diagnosis, and/or mitigation of disease or illness (e.g., HMG co-A reductase inhibitors (statins) like rosuvastatin, nonsteroidal anti-inflammatory drugs like meloxicam, selective serotonin reuptake inhibitors like escitalopram, blood thinning agents like clopidogrel, steroids like prednisone, antipsychotics like aripiprazole and risperidone, analgesics like buprenorphine, antagonists like naloxone, montelukast, and memantine, cardiac glycosides like digoxin, alpha blockers like tamsulosin, cholesterol absorption inhibitors like ezetimibe, metabolites like colchicine, antihistamines like loratadine and cetirizine, opioids like loperamide, proton-pump inhibitors like omeprazole, anti(retro)viral agents like entecavir, dolutegravir, rilpivirine, and cabotegravir, antibiotics like doxycycline, ciprofloxacin, and azithromycin, anti-malarial agents, and synthroid/levothyroxine); substance abuse treatment (e.g., methadone and varenicline); family planning (e.g., hormonal contraception); performance enhancement (e.g., stimulants like caffeine); and nutrition and supplements (e.g., protein, folic acid, calcium, iodine, iron, zinc, thiamine, niacin, vitamin C, vitamin D, and other vitamin or mineral supplements).

In certain embodiments, the active substance is one or more specific therapeutic agents. As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Listings of examples of known therapeutic agents can be found, for example, in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005; and “Approved Drug Products with Therapeutic Equivalence and Evaluations,” published by the United States Food and Drug Administration (F.D.A.) (the “Orange Book”). Examples of drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. In certain embodiments, the therapeutic agent is a small molecule. Exemplary classes of therapeutic agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents, antihistamines, antimigraine drugs, hormones, prostaglandins, antimicrobials (including antibiotics, antifungals, antivirals, antiparasitics), antimuscarinics, anxioltyics, bacteriostatics, immunosuppressant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated into the drug delivery device. These may be vitamins, supplements such as calcium or biotin, or natural ingredients such as plant extracts or phytohormones.

In some embodiments, the therapeutic agent is one or more antimalarial drugs. Exemplary antimalarial drugs include quinine, lumefantrine, chloroquine, amodiaquine, pyrimethamine, proguanil, chlorproguanil-dapsone, sulfonamides such as sulfadoxine and sulfamethoxypyridazine, mefloquine, atovaquone, primaquine, halofantrine, doxycycline, clindamycin, artemisinin and artemisinin derivatives. In some embodiments, the antimalarial drug is artemisinin or a derivative thereof. Exemplary artemisinin derivatives include artemether, dihydroartemisinin, arteether and artesunate. In certain embodiments, the artemisinin derivative is artesunate.

In another embodiment, the therapeutic agent is an immunosuppressive agent. Exemplary immunosuppressive agents include glucocorticoids, cytostatics (such as alkylating agents, antimetabolites, and cytotoxic antibodies), antibodies (such as those directed against T-cell recepotors or Il-2 receptors), drugs acting on immunophilins (such as cyclosporine, tacrolimus, and sirolimus) and other drugs (such as interferons, opioids, TNF binding proteins, mycophenolate, and other small molecules such as fingolimod).

In certain embodiments, the therapeutic agent is a hormone or derivative thereof. Non-limiting examples of hormones include insulin, growth hormone (e.g., human growth hormone), vasopressin, melatonin, thyroxine, thyrotropin-releasing hormone, glycoprotein hormones (e.g., luteinzing hormone, follicle-stimulating hormone, thyroid-stimulating hormone), eicosanoids, estrogen, progestin, testosterone, estradiol, cortisol, adrenaline, and other steroids.

In some embodiments, the therapeutic agent is a small molecule drug having molecular weight less than about 2500 Daltons, less than about 2000 Daltons, less than about 1500 Daltons, less than about 1000 Daltons, less than about 750 Daltons, less than about 500 Daltons, less or than about 400 Daltons. In some cases, the therapeutic agent is a small molecule drug having molecular weight between 200 Daltons and 400 Daltons, between 400 Daltons and 1000 Daltons, or between 500 Daltons and 2500 Daltons.

In some embodiments, the therapeutic agent is selected from the group consisting of active pharmaceutical agents such as insulin, nucleic acids, peptides, bacteriophage, DNA, mRNA, human growth hormone, monoclonal antibodies, adalimumab, epinephrine, GLP-1 Receptor agoinists, semaglutide, liraglutide, dulaglitide, exenatide, factor VIII, small molecule drugs, progrstin, vaccines, subunit vaccines, recombinant vaccines, polysaccharide vaccines, and conjugate vaccines, toxoid vaccines, influenza vaccine, shingles vaccine, prevnar pneumonia vaccine, mmr vaccine, tetanus vaccine, hepatitis vaccine, HIV vaccine Ad4-env Clade C, HIV vaccine Ad4-mGag, dna vaccines, ma vaccines, etanercept, infliximab, filgastrim, glatiramer acetate, rituximab, bevacizumab, any molecule encapsulated in a nanoparticle, epinephrine, lysozyme, glucose-6-phosphate dehydrogenase, other enzymes, certolizumab pegol, ustekinumab, ixekizumab, golimumab, brodalumab, gusellu,ab, secikinumab, omalizumab, tnf-alpha inhibitors, interleukin inhibitors, vedolizumab, octreotide, teriperatide, crispr cas9, insulin glargine, insulin detemir, insulin lispro, insulin aspart, human insulin, antisense oligonucleotides, and ondansetron.

In an exemplary embodiment, the therapeutic agent is insulin.

In certain embodiments, the therapeutic agent is present in the tissue interfacing component at a concentration such that, upon release from the tissue interfacing component, the therapeutic agent elicits a therapeutic response.

In some cases, the therapeutic agent may be present at a concentration below a minimal concentration generally associated with an active therapeutic agent (e.g., at a microdose concentration). For example, in some embodiments, the tissue interfacing component comprises a first therapeutic agent (e.g., a steroid) at a relatively low dose (e.g., without wishing to be bound by theory, low doses of therapeutic agents such as steroids may mediate a subject's foreign body response(s) (e.g., in response to contact by a tissue interfacing components) at a location internal to a subject). In some embodiments, the concentration of the therapeutic agent is a microdose less than or equal to 100 μg and/or 30 nMol. In other embodiments, however, the therapeutic agent is not provided in a microdose and is present in one or more amounts listed above.

In some embodiments, the component described herein comprises two or more types of therapeutic agents.

Some embodiments of the capsules disclosed herein may carry and deliver two or more types of therapeutic, diagnostic, and/or enhancement agents. For instance, an inventive capsule can contain and be configured to deliver two or more therapeutic agents at the same time in combination for certain treatments. In an example covering peritoneal dialysis aspects as well as delivery of biologics, a capsule may contain and deliver both short- and long-acting insulin to provide pre-prandial and basal coverage (e.g., 1-20 units of short-acting insulin and 10-100 units of long-acting insulin for basal coverage). Alternatively, a capsule may contain and deliver both clavulonic acid (e.g., 125 mg) and amoxicillin (e.g., 250-875 mg) for synergistic and enhanced absorption to reduce the total dose. In yet another example, a capsule may contain both carbidopa (e.g., 10-25 mg) and levo-dopa (e.g., 100-250 mg) and deliver them in a way that enhances absorption, increases or maximize their effect in the central nervous system, and reduces the total dose for the desired effect.

Agents carried and delivered by an inventive capsule may also include combinations of an activator or enhancer and a drug, peptide, or biologic. Suitable combinations include but are not limited to semaglutide and salcaprozate sodium (enhancer) and insulin and sodium caprate (enhancer).

An inventive capsule can also be used to create a foam in a subject's gastrointestinal tract. Such a foam can be created by releasing one or more agents from the capsule and agitating the released agents with the capsule inside the gastrointestinal tract. Suitable foams include but are not limited to steroid forms, such as hydrocortisone and budesonide foams, that be used for treating intestinal inflammation.

Exemplary Capsule Enhancing Drug Delivery for Heightened Bioavailability

Oral drug delivery is challenged by poor small intestinal drug absorption, so that many drugs are administered through more cumbersome and expensive methods. Luminal mucus poses a predominant steric and dynamic barrier to absorption. The capsule may locally clear the mucus layer, enhance luminal mixing, and topically deposit the drug payload in the small intestine to enhance drug absorption. The capsule's mucus-clearing and churning movements are facilitated by surface features that interact with small intestinal plicae, villi, and mucus. For vancomycin, and insulin, small peptides, capsule delivery enhanced bioavailability 20-40 fold in ex vivo and in vivo swine models when compared to standard oral delivery (p<0.05). Insulin delivery via the capsule resulted in significant and therapeutic decreases in blood glucose (p<0.05), establishing its potential to facilitate oral delivery of drugs that are normally precluded by absorption limitations.

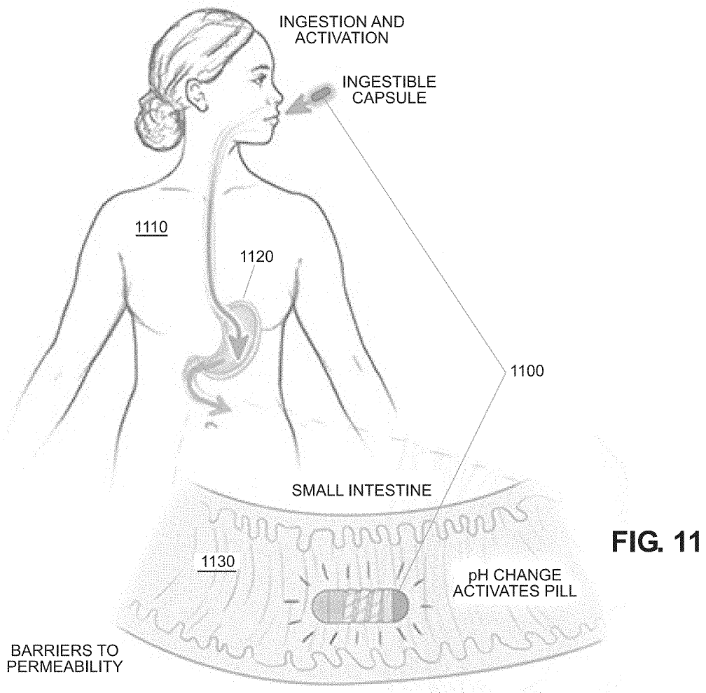

FIG. 11 shows the ingestion and activation of the capsule 1100. After ingestion by a user 1110, the capsule 1100 navigates through the user's gastrointestinal tract. The capsule may be activated using serial dissolution of pH-sensitive gelatinous membranes to expose surface features and close the circuit to activate the capsule. The capsule, sized as a triple-zero capsule, is orally ingested and carries onboard a drug payload volume up to 342.6 mm³ in its cargo hold (endcap). During passage through the stomach 1120, gastric fluid erodes away a gelatinous coating, which makes swallowing safe and comfortable, exposing the capsule's microtextured surface. Upon reaching the small intestine 1130, the pH of the intestinal fluid triggers a dissolvable activation membrane, closing an onboard circuit to start the capsule. Internal to the capsule, an offset weight laterally mounted on a motor generates a centrifugal force that induces rotational, oscillatory, and rocking movements of the capsule.

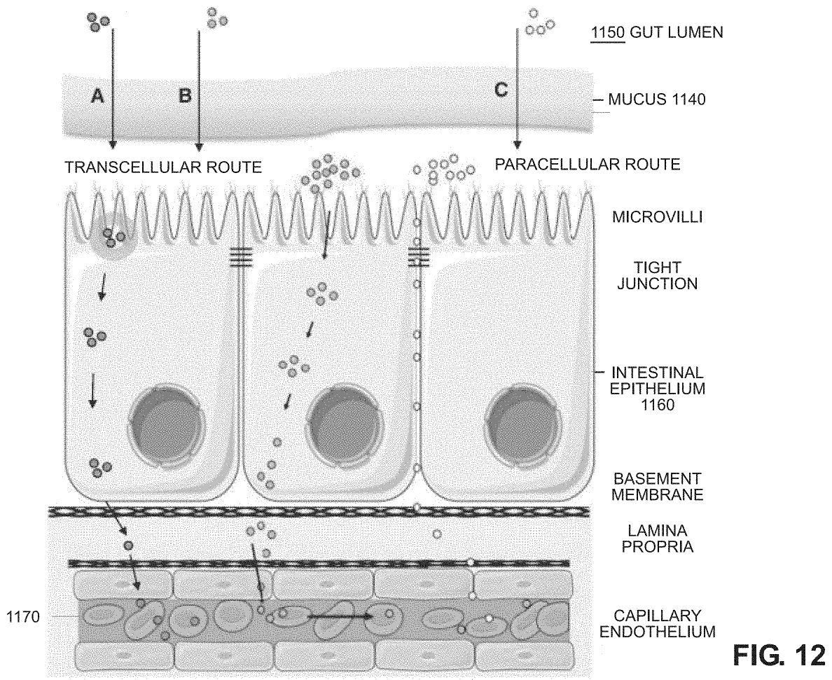

FIG. 12 shows conventional barriers to oral bioavailability due to low drug permeability in the small intestine. Through its viscous, hydrophilic, frequent turnover, and shear-thinning gel properties, mucus 1140 in the gut lumen 1150 serves as a dynamic, steric, and interactive barrier, preventing drugs in the lumen from reaching the surface of the intestinal epithelium 1160, through which the drug may be passed to the blood stream 1170. The capsule overcomes some of these challenges to increase oral bioavailability.

The capsule used a triple zero capsule's dimensions to aid oral administration. A central compartment in the capsule housed the battery, resistor, motor (1.5 V 3 V 6 mm×10 mm miniature micro vibrating coreless motor, A00000308) and offset weight. The circuitry in this compartment is closed upon dissolution of a polymer membrane which degrades at the pH of small intestinal fluid. This allows the pogo pin attached to the battery to contact the motor lead, thus closing the circuit. A secondary compartment houses the drug load and can be press fit onto the main compartment. A 1.55-volt, 80 mAh Silver Oxide battery was used due to its biocompatibility and its high capacity to size ratio. Prototypes were 3D printed using the VeroClear Photopolymer, selected for its biocompatibility, transparency, and chemical resistance. Capsules were thoroughly cleaned prior to administration. In preparation for assembly, the 3D printed parts were submerged in 2% sodium hydroxide solution and stirred for 15 minutes. The parts were then rinsed in de-ionized (DI) water four times before being left to dry.

Motor frequency was modulated through the use of resistors ranging from 0 to 120 ohms placed between the battery and the motor. The frequency of vibration was verified using a tachometer to measure the rotation rate of the offset weight over the period of 10 seconds.

The capsule underwent iteration of the surface geometry to enhance rotation and mucosal disruption. The baseline geometry utilized a smooth exterior shell similar to conventional triple-zero drug capsules. Protruding and intruding helical geometries were then added to increase the rotation rate of the capsule. Studded arrays along the spirals were in turn incorporated to increase the churning effect on the small intestine mucosal layer and further stimulate the villi for drug absorption. Due to the modular nature of the capsule, these features were incorporated and combined for fast prototyping of various geometries.

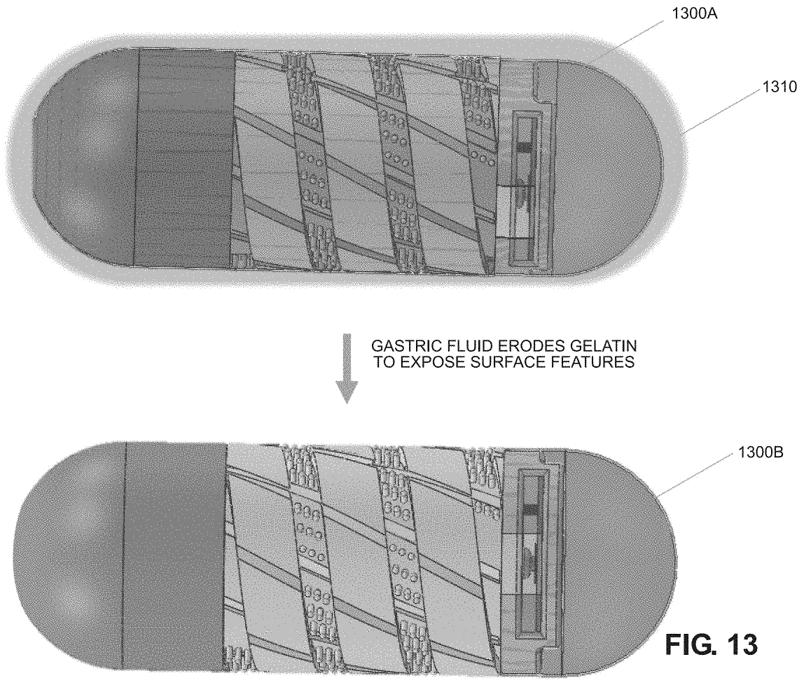

FIG. 13 shows the capsule 1300a having a coating 1310 disposed on the exterior surface of the capsule to cover the microtextural features and the drug payload. The coating 1310 dissolves or degrades in a fluid having a set pH (for example, the pH of gastric fluid or the pH of intestinal fluid in the small intestine). The dissolution or degradation of the coating 1310 reveals the capsule 1300b, exposing the microtextured features on the external surface of the capsule, exposing the drug payload, and/or opening the conduit to provide fluid communication with the membrane in the actuation assembly to actuate the capsule.

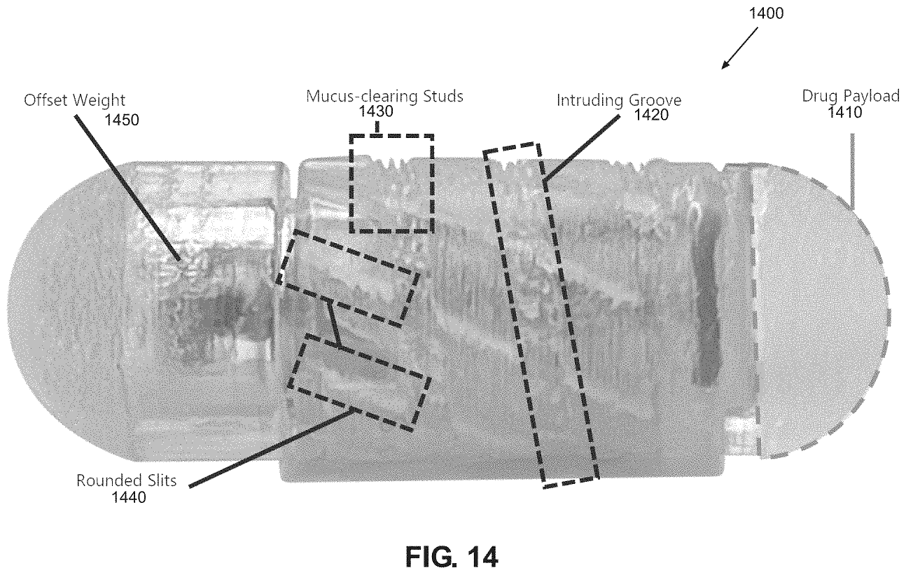

FIG. 14 shows a picture of the capsule 1400. The capsule 1400 includes a drug payload 1410 disposed in the endcap of the capsule. The surface of the housing of the capsule includes several forms of microtexturing, including an intruding groove 1420, mucus clearing studs 1430, and rounded slits 1440. The offset weight 1450 is visible inside the main cavity of the capsule.

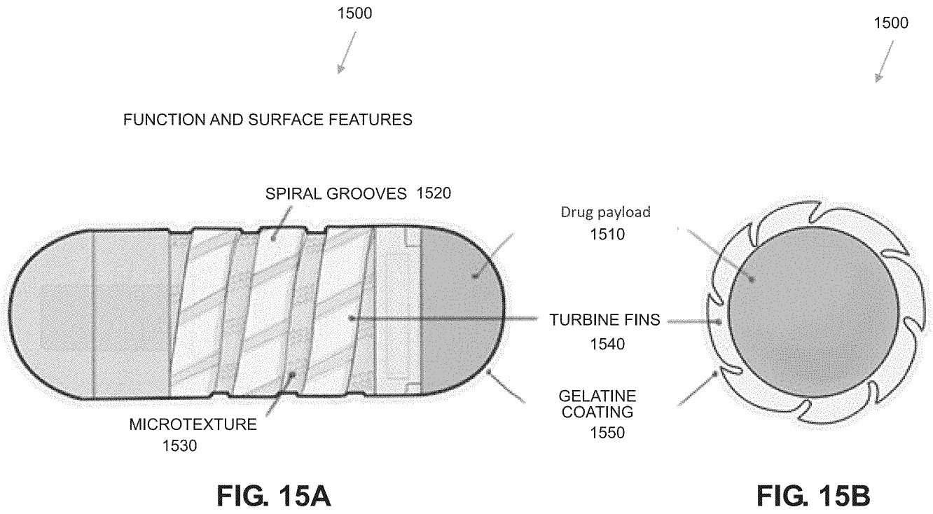

FIGS. 15A and 15B show additional views of a capsule 1500. The capsule 1500 includes a gelatin coating 1550 disposed over the whole capsule. The capsule 1500 includes a drug payload 1510 in the end cap of the capsule. The capsule housing includes several types of microtexturing, including spiral grooves 1520, studs 1530, and turbine fins 1540.

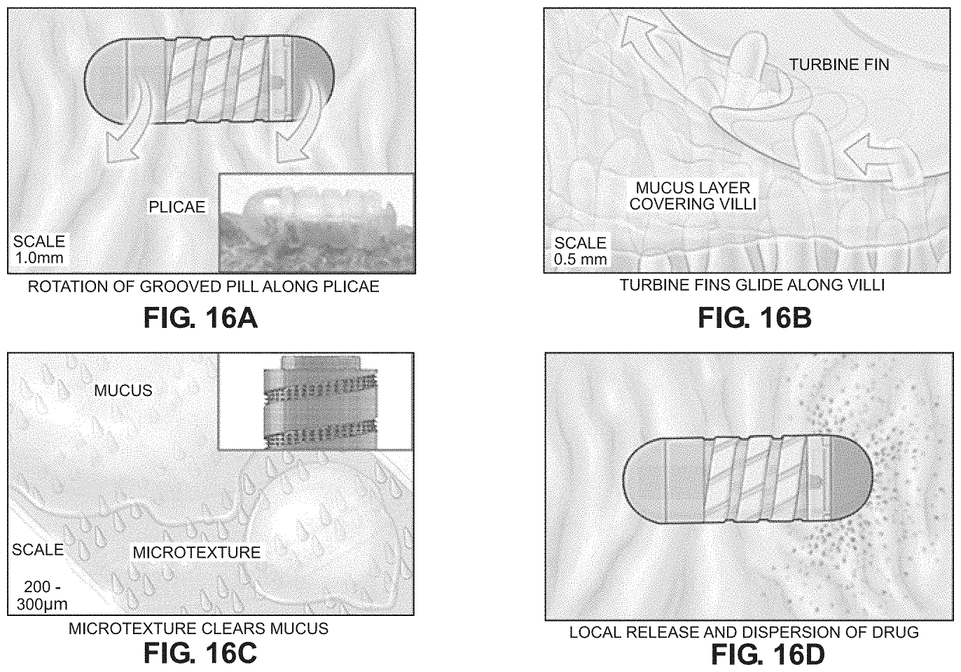

FIGS. 16A-16C show examples of microtextured features on the capsule interacting with the intestinal mucosa, and FIG. 16D shows the capsule releasing the payload in the small intestine. During its rotation, the capsule's surface features mechanically interact with the intestinal plicae, villi, and mucus to enhance drug delivery through various mechanisms. FIG. 16A shows the external helix (1.0 mm in width) provides substantial contact with plicae (1-10 mm). FIG. 16B shows the turbine fin rounded slits (0.5 mm) interfacing with villi (0.2-8 mm). Together the helix and rounded slits facilitate capsule rotation on the mucosa. The capsule's surface contour also increases mucosal surface contact wherein microtextured (200-300 μm) studs seated on the recessed surfaces of the helix churn and clear the 500-800 μm thick mucus layer coating the epithelium, as shown in FIG. 16C. FIG. 16D shows each rotation causing the release of the solid drug load via layer-by-layer erosion, thereby depositing drug particles. The capsule is active for 35 minutes and is moved along the GI tract by peristalsis whereby it is passed during defecation. The drug payload is positioned at one end of the capsule, allowing it to be easily manipulated by pharmacists, who can load any drug of choice. Additionally, the capsule's pH sensitivity can be tuned to serve other segments of the GI tract by modifying the dissolvable membrane properties.

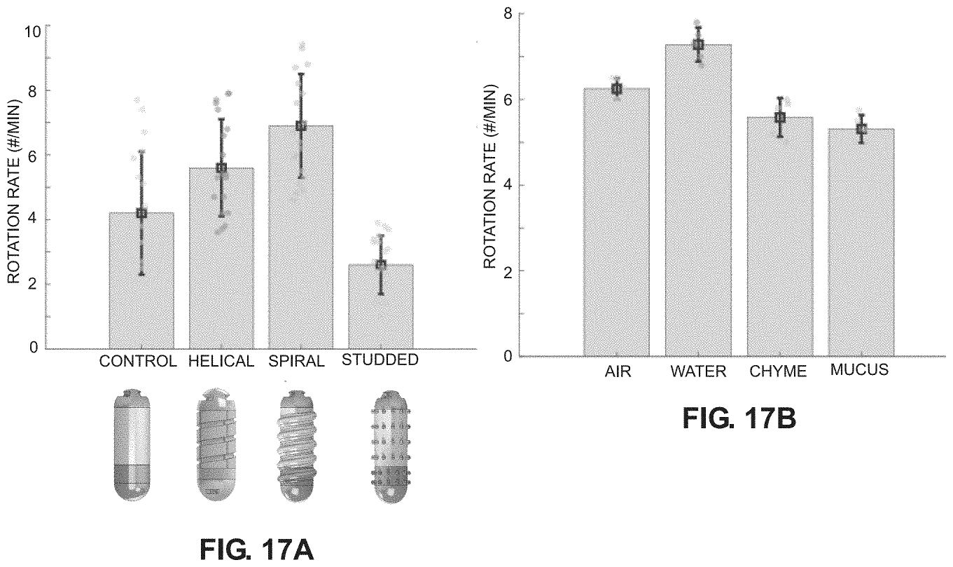

FIG. 17A shows rotation rates of microtextured capsules having different surface geometries on swine small intestine. To optimize rotation, surface geometries incorporating spiral, helical, and studded features were compared to a smooth exterior. Rotation rate was measured while the capsule rotated on freshly excised small intestinal tissue. Rotational rate was found to be greatest with a helical intrusion (6.9±1.6 rotations per minute (rpm)), likely due to alignment with plicae and accentuation of the oscillatory effect as compared to smooth (4.2±1.9 rpm), spiral extrusions (5.6±1.5 rpm), and studded exteriors (2.6±0.9 rpm) (n=20 trials for each). Thus, an outer body comprising helical intrusions was selected for the capsule.

FIG. 17B shows rotation rates of a microtextured capsule in different media. Rotation rate in air, water, chyme, and mucus was also assayed to provide insight on the range of rotation rates expected as the capsule encounters diverse media in the small intestine (n=5 trials each). Less than 30% variability was observed between media, indicating that the desired functionality would not be precluded even in the most viscous conditions.

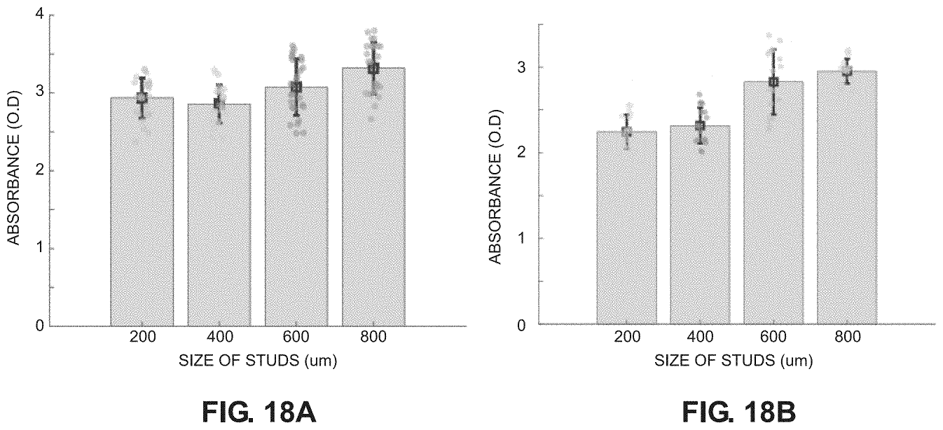

FIG. 18A shows optical absorbance of luminal fluid in a 4 cm segment of the intestine following 30 minutes of treatment with a microtextured capsule having studs of varying height. FIG. 18B shows optical absorbance quantification of mucus adhered to microtextured capsules following 30 minutes of rotation in swine small intestine with varying stud heights. In the recesses of the helical outer body, studs were fabricated to interrupt beds of mucus as the capsule strokes the surface. Studs of lengths ranging from 200 μm to 800 μm were assessed in their capability to wick and remove mucus. The surface contents of the capsule and luminal fluid following 20 minutes of operation in freshly excised small intestinal tissue were assessed with absorbance spectroscopy at 330 nm (n=9 trials/condition). 800 μm studs provided the greatest clearing and wicking of mucus. Inspired by torpedo blades and fins, rounded slits were designed in the helical outer body to generate propulsion of dislodged mucus into the luminal cavity and enhance mixing through turbulent flow.

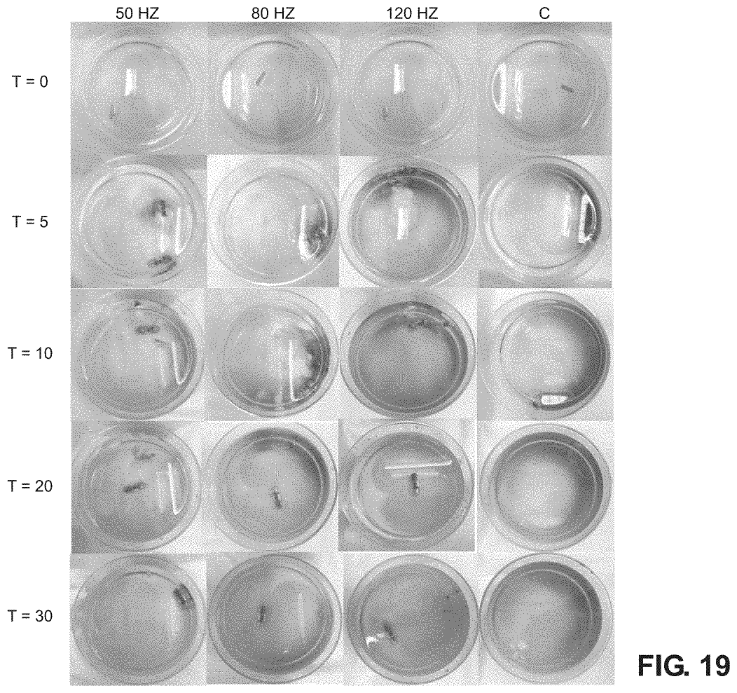

FIG. 19 shows mixing of a drug in a reaction chamber filled with a viscous mucus with a microtextured capsule at varying frequencies. The capsule's drug mixing capabilities were characterized by imaging the reaction chamber at 0, 5, 10, 20 and 30 minutes with the drug (darker powder) and the capsule operating at vibrational frequencies of 0 (control), 50 Hz, 80 Hz, and 120 Hz. Absorbance of samples from top, middle, and bottom of the chamber quantitively determined that the capsule provided faster dissolution of the drug and greater spatial dispersion. Frequencies of 80 Hz and 120 Hz performed better than 50 Hz. Given power considerations, 80 Hz was chosen as the operational frequency. Similar results were repeated using a swine small intestinal tissue, revealing consistent results.

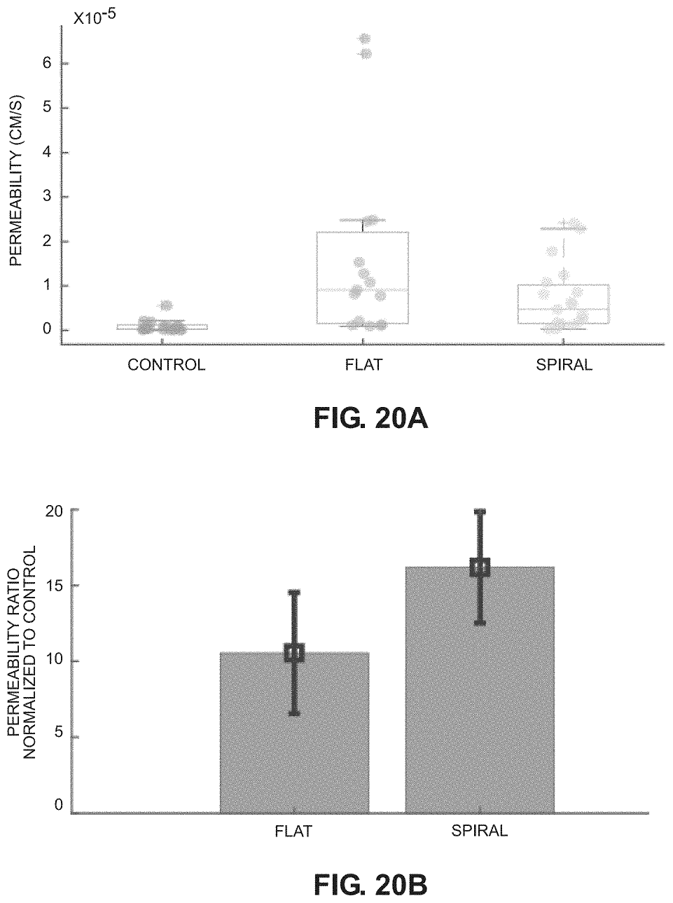

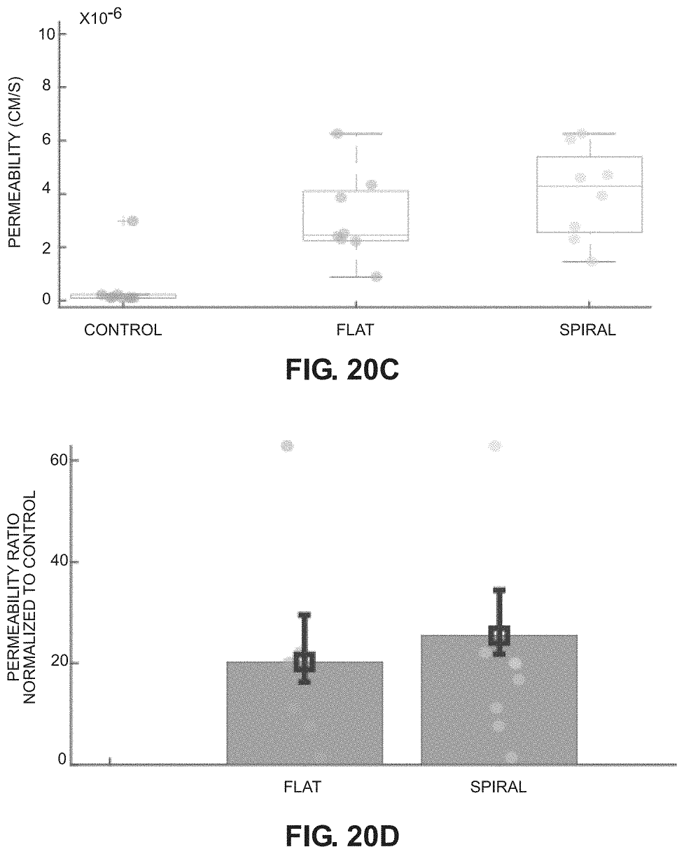

FIG. 20A shows drug permeabilities for vancomycin delivery with a smooth capsule (control) or microtextured capsule with flat or helical surface geometries in a Franz cell experiment on small intestinal swine tissue. A range of ex vivo and in vivo studies were performed to quantify the efficacy of the capsule in enhancing drug absorption. In FIG. 20A, using a Franz cell apparatus, vancomycin was delivered either by direct dilution in the donor well or through delivery with the capsule in the donor well. Both helical and smooth exteriors were assessed. Across 25 independent tissue samples deriving from n=5 animals, vancomycin drug permeability was observed to increase over 10-fold with capsule delivery as compared to controls (p<0.05, student's two-tailed heteroscedastic t-test). FIG. 20B shows drug permeabilities for vancomycin delivery normalized to their matched pair in the data shown in FIG. 20A. Given inter-animal variability of tissue properties, a ratio of permeability induced by the capsule to the control condition was calculated using a matched-pair format per animal. Capsules with a helical surface geometry significantly outperformed a smooth surface geometry (p<0.05, student's two-tailed heteroscedastic t-test).

FIG. 20C shows drug permeabilities for vancomycin delivery in swine small intestine by smooth capsule (control) or a helical or flat microtextured capsule. In anesthetized swine, sections of the small intestine were isolated to serve as independent testing sites while controlling for animal-specific properties such as hydration status, peristaltic rate, blood pressure, and perfusion. Vancomycin permeability was assessed through venous blood collection from the mesenteric plexus directly stemming from the isolated sections treated with either capsules or sham control pills carrying 100 mg vancomycin. Consistent with the Franz cell studies, capsules facilitated significantly greater tissue permeabilities, 20+ times the control (p<0.001, student's two-tailed heteroscedastic t-test). FIG. 20D shows drug permeabilities for vancomycin delivery normalized to their matched pair in the data shown in FIG. 20C. The helical surface additionally demonstrated a significant advantage over the smooth exterior. These results suggest that the capsule enhances absorption of small molecules such as vancomycin.

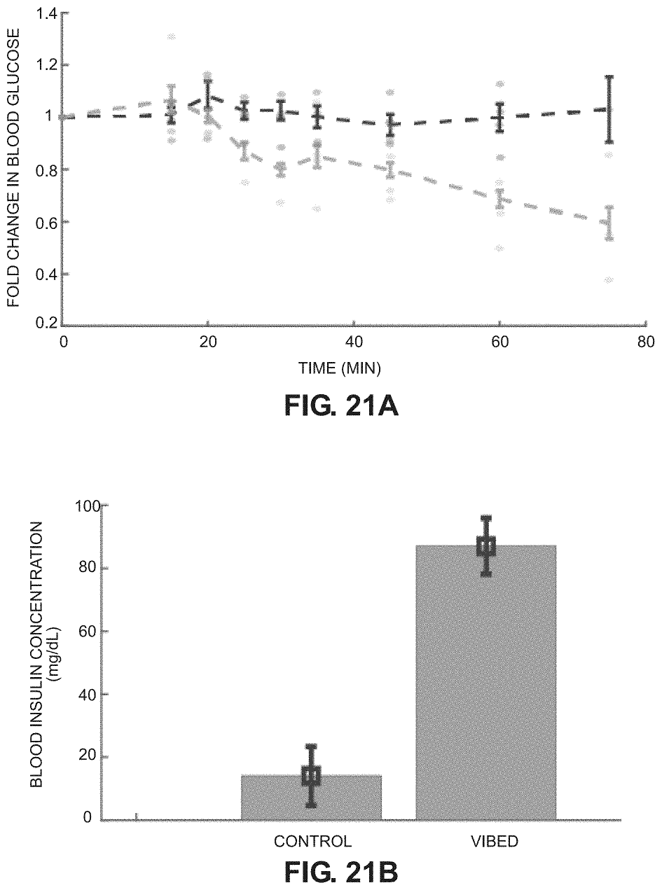

FIG. 21A shows plasma glucose measurements in swine following luminal insulin delivery (control, black) or delivery via microtextured capsule (experimental, red). FIG. 21B shows insulin concentration in blood measured 75 minutes after treatment with luminal insulin (control, upper trace/left column) or insulin via microtextured capsule (experimental, lower trace/right column). To assess the efficacy of the capsule to facilitate peptide drug delivery, oral insulin was delivered (100 units) via the capsule (n=7 animals) or endoscopic spray in the small intestine (control, n=5). Blood glucose and insulin concentrations were monitored for a 75-minute period with the drug delivery starting at 15 minutes. The capsule provided significantly greater bioavailability, causing a sharp decrease in plasma glucose levels (p<0.001, student's heteroscedastic t-test) and blood insulin levels (p<0.001, student's heteroscedastic t-test, n=5 animals) when compared to controls (n=5). Animals treated with the capsule demonstrated an average blood glucose reduction of 55.54±16.1 mg/dL, while controls demonstrated a variance of 16.6±17.3 mg/dL from baseline. When treated with the capsule, changes in plasma glucose levels were seen within 15 minutes and continued through the end of the monitoring period. In three animals, hypoglycemia (blood glucose<20 mg/dL) ensued at 60 minutes, necessitating dextrose infusion, and indicating a steady and significantly enhanced drug absorption.

Capsules were safely passed by the animal without complications, perforation, or obstruction in 10+ trials. No erosion of the mucosa, inflammation, infection, or hematological complications were sustained, as observed by endoscopy performed before and after capsule activity. The capsule was visualized radiographically passing through the animal alongside radiopaque (barium sulfate) beads placed to monitor the motility rate. The rate of clearance of the capsule was not significantly different from the passage of the radiopaque beads.

Histological analysis was performed on cross sectional samples from control (n=9) and stimulated (n=16) samples. Edema (control=1±0.707, stimulated=0.93±0.25) and inflammation (control=1.33±0.866, experimental=1.31±0.47) were insignificantly different between groups (p>0.1). While none of the control samples demonstrated vacuolization, 6 out of 16 in the experimental group demonstrated vacuolization, likely due to enhanced uptake.

To assay the capability of the capsule to assist in the delivery of larger molecules, fluorescein isothiocyanate-(FITC) dextran having various molecular weights were delivered using the capsule, at various motor frequencies, and compared to direct application (controls). The capsule was able to significantly increase uptake even with molecular weights as high as 150 kDa, although the greatest increases were seen at 40 kDa and 70 kDa. The frequency of the internal motor did not have a tractable impact on the rate of uptake.

This study demonstrates the utility of the capsule with microtextured surface features and different frequencies in enhancing oral drug absorption through localized drug delivery in regions with enhanced dispersion and clearing of mucus. Both ex vivo and in vivo testing consistently evinced a greater than 10-fold increase in drug permeability for both small molecule and peptide drug models. Capsule delivery of insulin resulted in a more gradual uptake as compared to the dynamics of subcutaneous or IV injection. Notwithstanding, it resulted in unanticipated hypoglycemia in 3 out of 7 animals, substantiating its potential to significantly enhance oral delivery of molecules that have previously seen little success. Increasing the efficacy of orally administered drugs with poor availability can in turn limit dosages and thereby increase safety and reduce cost.

Unlike other drug carrier systems such as lipid-based formulations or nanoparticles, the capsule maintains a long shelf-life and stability of the loaded drug and yields no biocompatibility concerns, as the robotics and electronic mechanisms remain sealed off and pass through the body after the drug is delivered.

Given the capsule's ability to rotate and create turbulent flow, it may be adapted for the administration of local anesthetics, such as lidocaine, for conditions such as irritable bowel syndrome in which topical application is required.

Dual Capsule Drug Delivery

A vibrating capsule can also be used to enhance delivery of a liquid therapeutic agent or a therapeutic agent carried by and released from one or more separate, non-vibrating capsules. The vibrating capsule can be ingested together with the liquid or separate, non-vibrating capsule(s). The capsules (or the capsule and the liquid) travel through the gastrointestinal tract to the stomach or intestine, where the vibrating capsule begins vibrating as described above and the other capsule releases its therapeutic agent. In some cases, the vibrating capsule carries additional therapeutic agent—either additional therapeutic agent or a different type of therapeutic agent—in which case, it releases its therapeutic agent as described above. In other cases, the vibrating capsule does not carry any therapeutic agent and instead simply vibrates against the interior wall of the gastrointestinal tract to enhance uptake of the therapeutic agent in the other capsule or the liquid therapeutic agent.

If desired, the vibrating capsule and other capsule(s) may contain magnets that attract the capsules to each other in order to ensure that the capsules reach the stomach or intestine at the (roughly) same time. The vibration and therapeutic agent release can be controlled passively, e.g., by dissolving pH-sensitive coatings or membranes as described above, or actively, e.g., using impedance-based proximity sensing, timers, or wireless communications. Using separate capsules (or a vibrating capsule and a liquid) to deliver therapeutic agents allows for a larger total drug payload. Moving the therapeutic agent off of the vibrating capsule can also simplify the design/decrease the complexity of the vibrating capsule.

### CONCLUSION

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain, using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An ingestible capsule comprising:
a housing forming a cavity and having a textured outer surface, the textured outer surface forming a helical depression and a plurality of protruding studs disposed in the helical depression;
a therapeutic agent disposed in or on the housing; and
a biodegradable coating on the textured outer surface of the housing, the biodegradable coating configured to dissolve in a fluid having a pH of 1.5 to 9.

2. The ingestible capsule of claim 1, wherein the textured outer surface includes a plurality of slits.

3. The ingestible capsule of claim 1, further comprising:
a motor disposed in the cavity and having a shaft; and
a weight mechanically coupled to the shaft and radially offset from a longitudinal axis of the shaft.

4. The ingestible capsule of claim 3, further comprising:
a power supply disposed in the cavity and electrically coupled to the motor.

5. The ingestible capsule of claim 4, further comprising one or more resistors disposed between the battery and the motor, to modulate a frequency of vibration of the motor.

6. The ingestible capsule of claim 3, wherein the shaft is configured to rotate about the longitudinal axis of the shaft at a frequency of about 2 Hz to about 400 Hz.

7. The ingestible capsule of claim 1, wherein the biodegradable coating is configured to dissolve in gastric fluid having a pH from about 1.5 to 3.5.

8. The ingestible capsule of claim 1, wherein the biodegradable coating is configured to dissolve in intestinal fluid having a pH from about 6 to 7.4.

9. The ingestible capsule of claim 1, wherein each protruding stud in the plurality of protruding studs has a diameter of about 200 μm to about 800 μm.

10. A method of delivering a therapeutic agent to a subject, the method comprising:
moving a portion of luminal mucus in the small intestine with an ingestible capsule by radially oscillating the ingestible capsule about a longitudinal axis of the ingestible capsule at a frequency of about 50 Hz to about 120 Hz; and
while moving the portion of luminal mucus, delivering a therapeutic agent from the ingestible capsule to the small intestine.

11. The method of claim 10, further comprising:
at least partially dissolving a biodegradable coating disposed on at least part of the ingestible capsule with stomach fluid.

12. The method of claim 10, further comprising:
at least partially dissolving a biodegradable coating disposed on at least part of the ingestible capsule with intestinal fluid.

13. The method of claim 12, wherein the biodegradable coating includes the therapeutic agent, such that the dissolving the biodegradable coating includes the delivering the therapeutic agent.

14. The method of claim 10, further comprising closing a circuit connecting a power supply and a vibrator in the ingestible capsule to induce the moving by dissolving, with intestinal fluid, a biodegradable insulating membrane disposed in electrical series between the power supply and the vibrator.

15. The method of claim 10, wherein the therapeutic agent has an oral bioavailability less than about 10%.

16. The method of claim 10, wherein the therapeutic agent has a molecular weight of about 10 kDa or less.

17. An ingestible capsule comprising:
a housing forming a cavity and having a textured outer surface;
a vibrator disposed in the cavity;
a power supply disposed in the cavity and configured to power the vibrator;
a therapeutic agent disposed in or on the housing; and
a biodegradable coating disposed on the textured outer surface of the housing, the biodegradable coating configured to dissolve in a fluid having a pH of 1.5 to 9, thereby exposing the therapeutic agent.

18. The ingestible capsule of claim 17, further comprising:
a biodegradable insulating membrane disposed in electrical series between the vibrator and the power supply and in fluid communication with an exterior of the housing, the biodegradable insulating membrane configured to dissolve in a fluid having a pH of about 2 to about 9, thereby closing a circuit connecting the power supply and the vibrator.

19. The ingestible capsule of claim 17, wherein the textured outer surface comprises at least one of a protrusion or depression.

20. The ingestible capsule of claim 19, wherein the at least one protrusion or depression includes a helical depression.

21. The ingestible capsule of claim 17, wherein the biodegradable coating comprises gelatin.

22. The ingestible capsule of claim 17, wherein the vibrator comprises:
a motor having a shaft; and
a weight mechanically coupled to the shaft and radially offset from a longitudinal axis of the shaft.

23. The ingestible capsule of claim 22, wherein the shaft is configured to rotate about the longitudinal axis of the shaft at a frequency of about 5 Hz to about 120 Hz.

24. The ingestible capsule of claim 22, wherein the shaft is configured to rotate about the longitudinal axis of the shaft at a frequency of about 80 Hz.
